(19) United States
(12) Patent Application Publication
(10) Pub. No.: US 2004/0073956 A1
(43) Pub. Date: Apr. 15, 2004

(54) Assay techniques based on growth stage dependent expression inc. elegans

(76) Inventors: Philippe Verwaerde, Neuville en Ferrain (FR); Thierry Bogaert, Kortrijk (BE)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/297,192

(22) PCT Filed: Jun. 8, 2001
(86) PCT No.: PCT/IB01/01213

(30) Foreign Application Priority Data
Jun. 8, 2000 (GB) 0014009.5

(51) Int. Cl. A01K 67/033

(52) U.S. Cl. 800/3; 800/8

(57) ABSTRACT

This invention is directed to new methods to perform assays with nematodes, and more particularly with microscopic nematodes such as C. elegans. In particular, the invention provides methods based on the use of growth-stage specific promoters to drive growth-stage specific gene expression.

This invention is directed to new methods to perform assays with nematodes, and more particularly with microscopic nematodes such as C. elegans.

The assay techniques described herein may inter alia be used for a variety of purposes, such as the discovery and development of compounds for pharmaceutical, veterinary and/or agrochemical use, the selection and isolation of mutant nematode strains, and may also be used for the specific expression of desired amino acid sequences, such as polypeptides and/or proteins, at various growth stages of the nematodes, among others.

Other aspects, embodiments, applications and advantages of the present invention will become clear from the further description hereinbelow.

General techniques and methodology for performing in vivo assays using the nematode worm Caenorhabditis elegans (C. elegans)—i.e. as a model organism for higher multicellular animals—have been described in the art, most notably in the following applications by applicant: PCT/EP99/09710 ( published on Jun. 15, 2000 as WO 00/34438); PCT/EP99/04718 (published on Jan. 15, 2000 as WO/00101846); PCT/IB00/00575 (published on Oct. 26, 2000 as WO 00/63427); PCT/IB00/00557 (published on Oct. 26, 2000 as WO 00/63425); PCT/IB00/00558 (published on Oct. 26, 2000 as WO 00/63426); as well as for instance PCT/US98/10080 (published on Nov. 19, 1998 as WO 98/51351), PCT/US99/13650, PCT/US99/01361 (published on Jul. 29, 1999 as WO99/37770), and PCT/EP00/05102.

As described in these applications, one of the main advantages of assays involving the use of C. elegans is that such assays can be carried out in multi-well plate format (with each well usually containing a sample of between 2 and 100 worms) and—also because of this—may also be carried out in an automated fashion, i.e. using suitable robotics (as are described in the aforementioned applications and/or as may be commercially available). This makes assays involving the use of C. elegans ideally suited for the screening of libraries of chemical compounds, in particular at medium to high throughput. Such automated screens may for instance be used in the discovery and/or development of new compounds (e.g. small molecules and/or small peptides) for pharmaceutical, veterinary or agrochemical/pesticidal (e.g. insecticidal and/or nematocidal) use.

Some other advantages associated with the use of C. elegans as a model organism (e.g. in the assay techniques referred to above) include, but are not limited to:

C. elegans has a short life-cycle of about 3 to 4 days. This not only means that these nematodes (and suitable mutants, transgenics and/or stable lines thereof) can be cultivated/generated quickly and in high numbers, but also allows assays using C. elegans to test, in a relatively short period of time and at high throughput, the nematode worms over one or more, and up to all, stages of life/development, and even over one or more generations. Also, because of this short life span, in C. elegans based-assays, compounds may be tested over one or more, and up to essentially all, stages of development, without any problems associated with compound stability and/or (bio)availability;

C. elegans is transparent, allowing—with advantage—for visual or non-visual inspection of internal organs and internal processes, and also the use of markers such as fluorescent reporter proteins, even while the worms are still alive. Also, as further mentioned below, such inspection may be carried out in automated fashion using suitable equipment such as plate readers;

C. elegans is a well-established and well-characterized model organism. For example, the genome of C. elegans has been fully sequenced, and also the complete lineage and cell interactions (for example of synapses) are known. In addition, C. elegans has full diploid genetics, and is capable of both sexual reproduction (e.g. for crossing) as well as reproduction as a self-fertilizing hermaphrodite. All this may provide many advantages, not only for the use of C. elegans in genetic and/or biological studies, but also for the use of C. elegans in the discovery, development and/or pharmacology of (candidate) drugs for human or animal use.

Techniques for transforming, handling, cultivating, maintaining and storing (e.g. as frozen samples, which offers great practical advantages) C. elegans are well established in the art, for instance from the handbooks referred to below. For example, C. elegans may be used as a one or more samples with essentially fully isogenic genotype(s).

Generally, in the assays described above, the nematodes are incubated in suitable vessel or container—such as a compartment or well of a multi-well plate—on a suitable medium (which may be a solid, semi-solid, viscous or liquid medium, with liquid and viscous media usually being preferred for assays in multi-well plate format). The nematodes are then contacted with the compound(s) to be tested, e.g. by adding the compound to the medium containing the worms. After a suitable incubation time (i.e. sufficient for the compound to have its effect—if any—on the nematodes), the worms are subjected to a suitable detection technique, i.e. to measure/determine a signal that is representative for the influence of the compound(s) to be tested on the nematode worms, which may then be used as a measure for the activity of the compound(s) in the in vivo assay. Often, such a signal will be based on and/or derived from (changes in) at least one biological, phenotypical, behavioural and/or biochemical property of the worm, such as drinking, pharynx pumping, movement, egg laying, mating or defecation (vide for instance PCT/IB00/00575). These properties are also generally referred to as “(biological) read outs” of or for the assay.

Often, in particular for automated assays, such a detection technique involves a non-visual detection method (as further described in the applications mentioned above), such as measurement of fluorescence or another optical method, measurement of a particular marker (either associated with worms or associated with the medium) such as an autonomous fluorescent proteins (AFP) for example green fluorescent protein (GFP), aequorin, alkaline phosphatase, luciferase, Beta-glucoronidase, Beta-lactamase, Beta-galactosidase, acetohydroxyacid, chloramphenicol acetyl transferase, horse radish peroxidase, nopaline synthase, or octapine synthase. For example, for automated assays carried out in multi-well plates, so called (multi-well) “plate readers” may be used for detecting/measuring such a signal.

For a further description of the above and other assay techniques involving the use of nematodes as a model organism, reference is made to the prior art, such as the applications by applicant referred to above.

For general information on C. elegans and techniques for handling this nematode worm, reference is made to the standard handbooks, such as W. B. Wood et al., “The nematode Caenorhabditis elegans”, Cold Spring Harbor Laboratory Press (1988) and D. L. Riddle et al., “C. ELEGANS II”, Cold Spring Harbor Laboratory Press (1997), and Caenorhabditis elegans, Modem Biological analysis of an organism: ed. by H. Epstein and D. Shakes, Methods in Cell Biology, Vol 48, 1995

Although the assay techniques described in the prior art mentioned above demonstrate the usefulness of C. elegans in a range variety of in vivo assays and for a variety of different purposes, there is an ever continuing need to develop further C. elegans based assays, in order to further broaden and expand the applicability of this model organism in drug discovery, development, testing and pharmacology.

The present invention provides such assay techniques, which, in addition to the advantages described hereinbelow, again have all the general advantages associated with the use of C. elegans as already described above.

In particular, the invention provides such assays, which are based on (changes in) growth and/or development of the nematode as the biological read out.

The invention is inter alia based on the fact that the nematodes used show a number of very distinct stages of development, e.g. from egg to the subsequent development stages referred to as embryonic (early, mid, late), L1, L2, L3 and L4, respectively, to adult. In addition, and mainly depending on environmental factors such as the absence of food, temperature, population and/or certain pheromones, the nematodes may optionally go into a specific and very distinctive stage called the “dauer-state” (which, although an optional stage of development, for the purposes of the present application is also considered a stage of life/development of the nematode).

Thus, more in particular, the present invention provides assay techniques which have been specifically designed to make use of such transition(s) by C. elegans from a first stage of development to another (i.e. second, and usually subsequent) stage of development as a biological read out.

The invention is also based on the fact that certain genes within the genome of the nematodes are expressed only during some of these stages of development of the nematodes, but not during some other stages. This is essentially because the promoters associated with these genes drive the expression of these genes in a manner that is dependent on the stage of development.

Some non-limiting examples of such “development-dependent” promoters, as well as the specific stage(s) of development in which they drive expression of their associated gene(s), are mentioned in Table 1 below. Others may be found in the handbooks referred to above.

TABLE 1
Promoters with growth stage dependent expression in C. elegans

| | | |
|---|---|---|
| glp-1 | Very early embryonic stage | WBG* 13(2):22 (Feb. 1, |
| | | 1994) |
| unc-54 | Mid-late embryonic stage | WBG 13(2):22 (Feb. 1, 1994) |
| myo-2 | Mid-late embryonic stage-adult | WBG 13(2):22 (Feb. 1, 1994) |
| vit-2 | Adult | WBG 13(2):22 (Feb. 1, 1994) |
| lin-28 | Embryonic-late L2 | WBG 14(5):56 (Feb. 1, 1997) |
| lin-4 | Late L1-adult | C. elegans II:501-518 |
| lin-14 | Late embryonic-mid L1 | WBG 11(3):46 |
| col-7 | L4-early adult | WBG (11)4:61 |
| col-19 | L4-early adult | WBG (11)4:61 |
| col-17 | Late embryonic-L3 | WBG (11)4:61 |
| ctl-1 | Dauer | Nature 399:162-166 |
| sod-3 | Dauer | FASEB 13:1385-1393 |

WBG*: worm breeders gazette

One promoter of particular interest for the purposes of the present invention is the vit-2 promoter, which specifically induces expression in the adult stage of the worm, and does so in a very stringent manner. The regulation and gene expression of the vitellogenin gene of C. elegans designated vit-2 promoter is well known, and the promoter region has been analyzed in detail. (MacMorris et al., Mol. Cell. Biol., 1992, 12:1652-1662; MacMorris et al., Mol. Cell. Biol., 1994, 14:484-491; Greenspoon et al., worm breeder's gazette, 1988, 10:25).

In the present invention, the “development-dependent” promoters referred to above are used to provide transgenic (strains of) nematode worms, which strains can be used in the assay techniques of the invention.

Thus, although promoters that may provide for development-dependant expression in C. elegans, as well as transgenic C. elegans lines that use such promoters for development-dependant expression in C. elegans have been described in the art, so far, such promoters and transgenes have not yet been used in the art in (the design of) assay techniques, in particular in (the design of) automated, high-throughput assay techniques.

Generally, to accomplish the present invention, the inventors have constructed transgenic nematodes which contain a growth stage dependent promoter operationally linked to a marker gene, and have used this transgenic nematode to develop assays which can be configured for a high throughput setting. The speed of growth or the passage in one of the growth stages which is monitored by the expression of the marker gene which is only expressed in a specific growth stage is then the criteria for selection. Mutant nematodes, and chemically treated nematodes are known to show growth delay, or even growth stage growth arrest. So this method allows for the selection of nematodes which growth faster or slower than the reference nematode. Particular descriptions and examples below are included to clarify this new method.

Thus, in a first aspect, the invention relates to a method for determining the influence of at least one exogenous factor on the development and/or growth of a sample of nematode worms, said method comprising:

a) providing a sample of nematode worms,

which nematode worms contain a marker gene operably linked to a promoter,

which promoter is capable of driving the expression of the marker gene in the nematode worms such that the marker gene is not expressed in at least a first development stage of the nematodes, but is expressed in at least a second development stage of the nematodes (different from the first life stage);

b) exposing said sample of nematode worms to at least one exogenous factor;

c) maintaining/cultivating said sample of nematode worms in a suitable medium, optionally over one or more life stages and/or generations;

d) subjecting the sample of nematode worms to at least one detection technique that is capable of detecting the signal generated by the marker gene (if expressed).

The nematodes used are preferably from the genus Caenorhabditis, such as Caenorhabditis briggsae or Caenorhabditis elegans.

The sample of nematodes may comprise any suitable number of worms, depending on the size of the container/vessel used. Usually, the sample will comprise between 2 and 500, in preferably between 3 and 300, more preferably between 5 and 200, even more preferably between 10 and 100 nematodes. When the assay is carried out in multi-well plate format, each well usually contains between 15 and 75 worms, such as 20 to 50 worms. Although not preferred, it is not excluded that a sample may consist of a single worm.

Usually, each such individual sample of worms will consist of worms that—at least at the start of the assay—are essentially the same, in that they are of the same strain, in that they contain the same mutation(s), in that they are essentially of an isogenic genotype, in that they show essentially the same phenotype(s), in that they are essentially “synchronised” (i.e. at essentially the same stage of development; it should however be noted that this stage of development may—and usually will—change during the course of the assay), in that they have been grown/cultivated in essentially the same way, and/or in that they have been grown under and/or exposed to essentially the same conditions, factors or compounds, including but not limited to pheromones, gene suppression (such as by RNAi), gene- or pathway-inducing factors or (small) molecules, and/or gene- or pathway-inhibiting factors or (small) molecules, and/or mutagenesis. However, in its broadest sense, the invention is not limited thereto.

In step a), when the sample of nematodes is initially provided, it is preferably such that the nematodes are essentially all in the first development stage.

Preferably said first development stage is such that it precedes the second development stage, in which said first development stage and said second development stage may or may not be separated (i.e. in time) by one or more further, intermediate development stages. For example, the first development stage may be L1, and the second development stage may be adult, with L2, L3, and L4 being intermediate development stages.

Preferably, the first development stage is chosen from eggs, an embryonal stage, L1, L2, L3, L4, or dauer; with eggs, embryonal stages, L1, L2 and dauer being particularly preferred, and L1 being the most preferred.

The second development stage is preferably a development stage subsequent to the first development stage (which may also be, if the first stage is dauer, any stage following escape from dauer) and is preferably chosen from L4, adult or dauer, and more preferably from adult or dauer, dependant on the choice of the first development stage. However, as can also be seen from Table 2 below, which lists some preferred combinations of first development stage, second development stage and promoter, the invention is not limited strictly thereto.

TABLE 2
some preferred combinations of first development stage, second
development stage and promoter.

| Promoter | First stage | Second stage |
|---|---|---|
| glp-1 | L1, L2, L3, L4, | very early embryonic stage (eggs) |
| | dauer, (adults) | |
| unc-54 | L1, L2, L3, L47 | mid-late embryonic stage (eggs) |
| | dauers, (adults) | |
| myo-2 | Very early eggs | mid-late embryonic stage-adult |
| vit-2 | Eggs, L1, L2, L3, | Adult |
| | (L4, dauer) | |
| lin-28 | L4, dauer, adult | Embryonic-late L2 |
| | (L3) | |
| lin-4 | Eggs | late L1-adult |
| lin-14 | L3, L4, adult, dauer, | late embryonic-mid L1 |
| | (L2) | |
| col-7 | Eggs, L1, L2, | L4-early adult |
| | dauer, (L3) | |
| col-19 | Eggs, L1, L2, | L4-early adult |
| | dauer, (L3) | |
| col-17 | Adult, dauer, (L4) | Late embryonic-L3 |
| ctl-1 | Eggs, L1, (L2, L3, | Dauer |
| | L4, adult) | |
| sod-3 | Eggs, L1, (L2, L3, | Dauer |
| | L4, adult) | |

In the assays of the invention, the nematodes may be kept in or on any suitable medium, including but not limited to solid and semi-solid media—but are preferably kept in a suitable liquid or viscous medium (e.g. with a viscosity at the temperature of the assay that is equal to a greater than the viscosity of M9 medium, as measured by a suitable technique, such as an Ubbelohde, Ostwald and/or Brookfield viscosimeter).

Generally, suitable media for growing/maintaining nematode worms will be clear to the skilled person, and include for example the media generally used in the art, such as M9 (10×M9 buffer: 30 g KH₂PO₄, 75.212 g Na₂HPO₄. 2H₂O, 50 g NaCl, 10 ml 1M MgSO₄, add up to 1 L), S-buffer (5.9 9 NaCl, 50 ml 1M KH₂PO₄, 1 ml 5 g/L cholesterol, add up to 1 L ), and the further media described in the applications and handbooks mentioned hereinabove.

The medium may further contain all factors, compounds and/or nutrients as may be required for the survival, maintenance and/or growth of the worms. For this, reference is again made to the prior art, such as the applications and handbooks referred to above. The medium may also contain a suitable source of food for the worms such as bacteria, for example a suitable strain of E. coli in a suitable amount, e.g. between 0.001 and 10% (w/v), preferably between 0.01 and 1%, more preferably between 0.1 and 0.2%, such as about 0.125% w/v. In one specific embodiment, further described below, said bacteria may also contain or express a double stranded RNA (construct), intended for specific gene down regulation in the nematode worm, e.g. by means of RNA-interference (vide PCT/EP99/04718)

The assay may be carried out at a suitable temperature, which may for example be a temperature of between 10° C. and 30° C., preferably between 20° C. and 27° C., such as 21, 22, 23, 24, 25 or 26° C., depending on the specific strain used. The temperature may be kept essentially constant during the course of the assay, and/or may be varied, e.g. within the ranges indicated above.

In the method of the invention, the sample of nematodes can be kept—e.g. maintained, grown or incubated—in any suitable vessel or container, but is preferably kept in a well of a multi-well plate, such as a standard 6, 24, 48, 96, 384, 1536, or 3072 well-plate (in which each well of the multi-well plate may contain a separate sample of worms, which may be the same or different). Such plates and general techniques and apparatus for maintaining/ handling nematode worms in such multi-well plate format are well known in the art, for instance from the applications mentioned hereinabove.

The method/assay of the invention is preferably carried out in an automated fashion, e.g. using the equipment and techniques described in the applications mentioned above.

In the invention, a nematode strain is used that contains a marker gene that is operably linked to a promoter, which promoter is capable of driving the expression of the marker gene in the nematode worm(s) such that the marker gene is not expressed in at least a first development stage of the nematodes, but is expressed in at least a second development stage of the nematodes (different from the first development stage).

As already indicated hereinabove, such promoters are also referred to herein as “development-dependent” promoters, and some preferred examples have been given above.

A particularly preferred development-dependent promoter is the vit-2 promoter. An operational fusion of a DNA sequence (gene, cDNA) with the vit-2 promoter allows for the expression of this DNA sequence in the adult stage of C. elegans, and not in the other life stages of C. elegans such as the L1, L2, L3, and L4 larvae stages and the dauer stages.

In the present disclosure, two or more nucleotide sequences, such as a promoter and a marker gene, are considered “operably linked” when they are in a functional relationship with each other. For instance, the development-dependent promoter is considered “operably linked” to the marker gene if said promoter is able to initiate or otherwise control/regulate the transcription and/or the expression of said marker gene, in particular in a development-dependent manner (and in which said marker gene should be understood as being “under the control of” said promoter). Generally, when two nucleotide sequences are operably linked, they will be in the same orientation and usually also in the same reading frame. They will usually also be essentially contiguous, although this may also not be required.

The marker gene may be any gene which, upon expression in C. elegans—i.e. under the control of the development-dependent promoter—provides a signal that can be detected, e.g. visually or preferably by the automated, non-visual detection techniques referred to above.

For example, the marker gene may be chosen from green fluorescent protein, beta-galactosidase, beta-lactamase, luciferase, acetohydroxyacid synthase, alkaline phosphatase, beta-glucuronidse, chloramphenicol acetyltransferase, horseradish peroxidase, nopaline synthase and/or octapine synthase. Other suitable marker genes will be clear to the skilled person, and are for instance described in the applications referred to above.

In a specific embodiment, the gene may be a toxic gene, e.g. a gene that encodes a gene product that is toxic (e.g. lethal) to the nematode. Thus, another application of the invention consists in the conditional expression of putative toxic genes, and in the conditional expression genes, to be expressed in specific growth stage in nematodes such as C. elegans. When toxic genes are expressed in nematode at any growth stage, and surely in the early development of the nematode, this will have dramatic influences on the further development and vitality of the nematode. It may be opportune to express such genes in a particular growth phase of the worm, such as the L1, L2, L3, L4, adult or dauer stages. Such transgenic nematodes have more change to survive the expression of the toxic gene and may be used for further analysis, for instance in a HTS assay, screening for compounds, mutants, etc. Some preferred, but non-limiting examples of such toxic genes are ataxin, alpha-synuclein, ubiquitin, the tau gene, the huntington gene, the best macular dystrophy gene product, unc-53; others are mentioned in the applications referred to above.

The nematode strain used in the invention may generally be provided by transforming a suitable nematode strain with a nucleotide sequence that comprises the marker gene under the control of the development-dependent promoter. Preferably, said nucleic acid sequence is in the form of a genetic construct, which may be DNA or RNA (and are preferably double-stranded DNA) and which is preferably in a form suitable for transformation of the nematode strain used. For example, it may be in the form of a construct that, upon transformation, is integrated in the genomic DNA of the nematode, and/or may be in a form suitable for independent replication, maintenance and/or inheritance in the nematode. Preferably, the construct is also such that it is capable of independent replication, maintenance and/or inheritance in the (micro-) organism used for cloning, such as E. coli. For instance, said genetic construct may be in the form of a plasmid, vector, viron or transposon.

The genetic construct(s) used in the invention may further contain—i.e. besides the nucleotide sequences encoding the development-dependent promoter and the marker gene—one or more further suitable elements of genetic constructs known per se, including but not limited to selection markers and/or elements that may facilitate or increase (the rate of) transformation or integration. These and other suitable elements for such genetic constructs will be clear to the skilled person, also from the applications referred to above.

The constructs of the invention can be provided in a manner known per se, which will generally involve techniques such as restricting and linking nucleic acids/nucleic acid sequences, as will be dear to the skilled person. Reference is made to the standard handbooks, such as Sambrook et al, “Molecular Cloning: A Laboratory Manual” (2nd. ed.), Vols. 1-3, Cold Spring Harbor Laboratory Press (1989) and F. Ausubel et al, eds., “Current protocols in molecular biology”, Green Publishing and Wiley Interscience, New York (1987). The nucleic acids encoding the development-dependent promoters and marker genes used in the invention have been described in the art and can be provided in the manner described therein.

The nematodes may be transformed with the constructs in any suitable manner, such as micro-injection or ballistic transformation, for which reference is made to the handbooks referred to above, as well as for instance in PCT/EP99/01903 ( published as WO 99/49066)

The nematode strain that is transformed with the nucleotide sequence encoding the marker gene/development-dependent promoter—i.e. to provide a nematode strain useful in the assay of the invention—is not particularly limited, and may for instance be any nematode strain known per se, such as wildtype, N2 or hawaiian (CB4856, Hodgkin et al., Genetics 146:149-164, 1997). Also, specific mutant nematode strains or lines and transgenic strains or lines may be used which are particularly suited/adapted for transformation and/or the specific transformation technique used, or if they are desired in the assay.

In one embodiment, before use in the present assays, the nematodes are subjected to random or specific mutagenesis. Thereupon, the different strains resulting from the mutagenesis may be tested in the assay(s) of the invention, and optionally may be compared to the original strain and/or to a(nother) reference strain. This may be done with and/or without exposure to the exogenous factor(s) and may for instance be used to identify genes and/or mutations that influence the development and/or growth of the nematodes, and/or to identify genes and/or mutations which alter or influence the response of the nematodes (i.e. with respect to development and growth) to the exogenous factors. For example, when a mutation in a gene leads to a marked change in development and/or growth (as determined using the assay(s) of the invention), or leads to a markedly different response to the exogenous compound(s), it may be concluded that said gene is involved in development or growth and/or in the response of the nematode to the exogenous factor(s). In this way, the assays of the invention may for instance be used to determine the function of (known or unknown) genes (for instance as part of a functional genomics program) and/or to determine the mode of action of the exogenous factor(s).

In step b), a sample of nematodes containing the marker gene under the control of the development-dependent promoter is exposed to the exogenous factor(s) to be tested. This may be carried out while the nematodes in the sample are (still) in the first stage of development, and/or in any subsequent stage(s) of development. Preferably, however, the sample of nematodes is exposed to the at least one exogenous factor in at least one stage of development which precedes the second stage of development (however, it should be noted that the invention does not exclude that the sample of nematodes is still in contact with the exogenous factor(s) while the nematodes transit into and/or are in the second stage of development).

For example, the nematodes may be exposed to the exogenous factor(s) in only a single stage of development (such as only in the first stage or only in a subsequent stage that precedes the second development stage), in two or more stages (which may include the first stage, any subsequent stage(s) and/or the second stage), or essentially continuously throughout the duration of the assay.

Thus, generally, the nematodes may be exposed to the exogenous factor(s) during a time of 1 minute up to the entire life (cycle) of the nematodes, and/orto the duration of the assay. Usually, a contact time of between 5 minute and 110 hours, preferably between 10 minutes and 80 hours will be preferred.

The total time for the assay will preferably be such that it is sufficient to allow at least one of the nematodes in the sample to transit from the first development stage into a subsequent development stage, and more preferably sufficient to allow at least one of the nematode worms in the sample to enter from the development stage into the second development stage, optionally via any (further) intermittent stages of development

For example, in step c), the sample of nematode worms may be maintained/cultivated for a time such that at least 1%, preferably at least 5%, of the nematode worms present in the sample enter from the first development stage into at least one other/further development stage.

Also, for example, in step c), the sample of nematode worms may be maintained/cultivated for a time such that at least 1%, preferably at least 5%, of the nematode worms present in the sample enter from the first development stage into the second development stage.

Often, the total time for the assay will be at least such that it would allow at least one of the nematode worms present in a reference sample—i.e. a sample not containing any exogenous factor(s)—to enter from the first development stage into the second development stage, optionally via any (further) intermittent stages of development.

For example, for assays from the following first development stage to the following second development stage, the total time of the assay can be as follows: from eggs to adults: 45 to 110 hours; from L1 to adults: 30 to 80 hours; from eggs to L1: 13 to 30 hours; from L1 to L2: 13 to 25 hours; from L2 to L3: 8 to 20 hours; from L3 to L4: 8 to 15 hours; from L4 to adult: 8 to 25 hours; for assays involving dauer as the first or second stage: between 8 and 72 hours (depending on the strain used, temperature and food quality, nematodes will generally enter the L1 growth stage between 13 and 30 hours, the L2 growth stage between 24 and 55 hours, the L3 growth stage between 30 and 70 hours, the L4 growth stage between 38 and 85 hours, and the adult stage between 45 and 110 hours, starting from eggs).

During the duration of the assay, the sample may be subjected to the—preferably non-visual—detection method for determining/measuring the expression of the marker gene essentially continuously during the entire duration of the assay, essentially continuously during one part of the duration of the assay (usually the latter part, when the nematodes are considered likely to enter the second stage of development, e.g. during the last 24, 12, or even 6 hours of the duration of the assay), at regular intervals, or any combination thereof.

In the assays of the invention, each individual sample of nematode worms will generally be exposed to a single exogenous factor to be tested, at a single amount or concentration; with different samples (e.g. as present in the different wells of the multi-well plate used) being exposed either to different concentrations of the same factor (e.g. to establish a dose response curve for said factor), to one or more different factors (e.g. in the case of compounds for instance are part of a chemical library and/or of a chemical class or series, such as a series of closely related structural analogues; or in case of a library or series of dsRNA constructs for RNAi), or both (e.g. to the same and/or different factors at different concentrations).

It is also within the scope of the invention to expose the (sample of) nematodes to two or more factors—at essentially the same time or sequentially (e.g. with an intermediate washing step)—for example to determine whether the two factors have an effect which is the same or different from both the factors separately (e.g. to provide a synergistic effect or an inhibitory or competitive effect).

Furthermore, it is within the scope of the invention to use one or more reference samples, e.g. samples without any factor(s) present, and/or with a predetermined amount of a reference factor. The invention also includes the use, in an assay, of two or more samples of nematode worms of different strains (e.g. each containing a marker gene under the control of a (different) development-dependent promoter), e.g. to compare (the effect of th factors(s) to be tested on) said different strains.

In one specific embodiment, which is referred to herein as an “FPTP-type assays”, each sample of a series of two or more essentially similar samples of nematode worms (e.g. containing the same development-dependant promoter, preferably the same marker gene—although this is not strictly required—and preferably comprised of worms in the same stage of development) is exposed, in essentially the same manner (e.g. time and conditions, but optionally at different concentrations), to (a) different exogenous factor(s), and optionally to one or more reference factors. Thereupon, the order in which the nematodes present in each of these samples enter the second development stage is determined, i.e. by determining the order in which the samples of the series show expression of the marker gene (i.e. which sample shows the expression of the marker gene first, second, third, etc.). Inter alia, this allows the different factors present in each of the samples to be compared and/or ranked according to their influence on the development/growth of the nematode, and also compared to the reference factor(s). This for instance allows the identification of factors with an influence on the nematodes comparable to, or even improved compared to, the influence of the reference factors. Generally, such FPTP-assays will involve determining the (possible) expression of the marker gene in the series of samples essentially continuously, at least during the last 36, 24, 12, or 6 hours of the assay.

Thus, in a specific embodiment, the invention relates to a method for determining the influence of at least a first exogenous factor on the development and/or growth of a sample of nematode worms, said method comprising:

a) providing at least a first and a second sample of nematode worms,

in which the nematode worms in each sample contain a marker gene operably linked to a promoter,

which promoter is capable of driving the expression of the marker gene in the nematode worms such that the marker gene is not expressed in at least a first development stage of the nematodes, but is expressed in at least a second development stage of the nematodes (different from the first life stage);

b) exposing at least said first sample of nematode worms to said first one exogenous factor;

c) maintaining/cultivating said samples of nematode worms in a suitable medium, optionally over one or more life stages and/or generations;

d) subjecting the samples of nematode worms to at least one detection technique that is capable of detecting the signal generated by the marker gene (if expressed);

e) determining the time required for the first sample of nematode worms to show expression of the marker gene (as determined by the signal detected for the first sample in step b)), and preferably also determining the time required for the second sample of nematode worms to show expression of the marker gene (as determined by the signal detected for the second sample in step b)); and/or comparing the time required for the first sample of nematode worms to show expression of the marker gene with the time required for the second sample of nematode worms to show expression of the marker gene.

In one aspect, the second sample of nematode worms will be a reference sample, e.g. a sample of worms that is not exposed to any exogenous factor, or to a known reference factor. The second sample may also be exposed to a second exogenous factor, e.g. to compare the first and the second factor.

Generally, as already indicated above, the assay according to this aspect, of the invention will involve the use/testing of a series of samples, e.g. more than 5, preferably more than 10, such as about 6, 24, 48, 96, 384, 1536, or 3072 (i.e. essentially the number of wells of a multi-well plate), each sample being exposed to a different factor and/or to a different concentration of factor (including any reference samples), and the samples than being ranked as described above.

Usually, to allow for a good comparison between the samples/factors, all samples will be essentially similar (as described above) and cultivated/maintained in an essentially similar manner. These FPTP-assays may further be carried out in essentially the manner described herein.

In all the assays described above, the exogenous factor may be any factor the influence of which on the growth/development of nematode worms is to be tested. The exogenous factors may for instance be chosen from small compounds (as defined below), small peptides (as defined below), factors which induce or suppress specific pathways in the worm, factors which induce or suppress (the expression of) specific genes in the worm (such as dsRNAi for RNA-interference), polypeptide and/or proteins, or extracts from natural products( such as plants, animals, fungi, bacteria), amino acids and derivatives, hormones and derivatives, nucleic acids and derivatives.

For the purposes of the present disclosure, a “small molecule” generally means a molecular entity with a molecular weight of less than 1500, preferably less than 1000. This may for example be an organic, inorganic or organometallic molecule, which may also be in the form or a suitable salt, such as a water-soluble salt.

The term “small molecule” also covers complexes, chelates and similar molecular entities, as long as their (total) molecular weight is in the range indicated above.

In a preferred embodiment, such a “small molecule” has been designed according, and/or meets the criteria of, at least one, preferably at least any two, more preferably at least any three, and up to all of the so-called Lipinski rules for drug likeness prediction (vide Lipinksi et al., Advanced Drug Delivery Reviews 23 (1997), pages 3-25). As is known in the art, small molecules which meet these criteria are particularly suited (as starting points) for the (design and/or) development of drugs (e.g) for human use, e.g. for use in (the design and/or compiling of) chemical libraries for (high throughput screening), (as starting points for) hits-to-leads chemistry, and/or (as starting points for) lead development.

In a preferred embodiment, such a “small molecule” has been designed according, and/or meets the criteria of, at least one, preferably at least any two, more preferably at least any three, and up to all of the so-called Lipinski rules for rational drug design (vide Lipinksi et al., Advanced Drug Delivery Reviews 23 (1997), pages 3-25). As is known in the art, small molecules which meet these criteria are particularly suited (as starting points for) the design and/or development of drugs (e.g) for human use

Also, for these purposes, the design of such small molecules (as well as the design of libraries consisting of such small molecules) preferably also takes into account the presence of pharmacophore points, for example according to the methods described by I. Muegge et al., J. Med. Chem. 44, 12 (2001), pages 1-6 and the documents cited herein.

The term “small peptide” generally covers (oligo)peptides that contain a total of between 2 and 35, such as for example between 3 and 25, amino acids (e.g. in one or more connected chains, and preferably a single chain). It will be clear that some of these small peptides will also be included in the term small molecule as used herein, depending on their molecular weight.

Thus, the methods of the invention may in particular be used to test and/or screen (libraries of) such small molecules and/or peptides, in the manner as further outlined herein.

According to another embodiment, the exogenous factor is a factor that suppresses or enhances the expression of one or more genes in the nematodes used. In one preferred example, this factor may be a dsRNA, which may be used for gene suppression in accordance with well-known RNA-interference techniques. Such dsRNA may for instance be provided to the nematode worms in the manner described in PCT/EP99/04718 (published as WO 00/01846) or PCT/US98/27233 (published as WO 99/32619), e.g. by injection of dsRNA or by feeding of bacteria containing/expressing the dsRNA to the nematode. In this latter embodiment, for example, the effect(s) of the suppression of one or more gene(s) on the growth or development of the nematode worms and/or on the response of other exogenous factors, may be determined.

The nematodes may be exposed to the exogenous factor in any suitable manner, such as by incorporating the exogenous factor in the medium in which the nematode worms are grown/maintained or by incorporating the nematode worms in the food of the nematodes (e.g. in the case of dsRNA for RNAi purposes).

The nematode worms may take up the exogenous factor in any suitable manner, such as by drinking, feeding, soaking, pharynx pumping, or in any other suitable way, e.g. either through (a part of) the gastrointestinal tract, the cuticle and/or through openings in the cuticle, and either through an active or passive uptake mechanism, or any combination thereof.

When the exogenous factor is a compound, it will usually be used in step b) at a concentration of between 0.1 nanomolar and 100 milimolar, preferably between between 1 nanomolar and 50 milimolar, more preferably between 10 nanomolar and 10 milimolar, even more preferably between 100 nanomolar and 5 milimolar, in particular between 1 micromolar and 1 milimolar, even more particular between 10 micromolar and 600 micromolar, most particular between 20 micromolar and 500 micromolar, such as about 30 micromolar for compound selection screens and about 300 micromolar for compound resistance screens.

For dsRNA, suitable amounts will be as described in the PCT/EP99/04718 (published as WO 00/01846) or PCT/US98/27233 (published as WO 99/32619).

The assay techniques of the invention may be used for several different applications, some non-limiting examples of which will now be further described.

A first application is to identify and select chemical entities that may be used in the development of pharmaceutical products, veterinary products, and pesticides. In this respect, it should also be noted that the invention may not just be used to identify exogenous factors (such as compounds) which directly influence development and/or growth, but also compounds which influence other behavioural, biological, phenotypical and/or biochemical processes which in turn influence growth and/or development, such as metabolic processes, feeding/drinking behaviour and/or (other) processes which are controlled by the central nervous system or other nerve cells.

Thus, the invention may also be used to identify compounds which may influence metabolic processes and neuron-controlled processes, not just in nematodes, but also in higher animals including humans and other mammals, for which the nematode is used as a model organism. Thus, the assays of the invention may be used in the discovery and/or development of pharmaceuticals and/or veterinary products.

Also, exogenous factors such as compounds which, in the assays of the invention, retard growth and/or development may find use in the development of novel insecticides or other pesticides (including but not limited to nematocides).

Another application is to identify and select new mutants, and further on isolating the genes which are mutated. This genes and the proteins they encode for are then considered as putative target genes and/or members of biochemical pathways. In a specific variant of this objective, mutants are selected that show resistance to a chemical compound, and once again the final objective is to isolate the mutated gene.

A third possible application is related to the isolation of genes, and the proteins they encode for by dsRNA inhibition (RNAi). The isolated genes and the proteins they encode for are considered as putative target genes, members of biochemical pathways, resistance.

In the development and performance of HTS assays with nematodes, the synchronicity of the animals is of major importance, i.e. nematodes used in the assay need to be at the same growth stage. Although several methods have been developed to grow a culture of nematodes at the same speed, while they are in the same growth stage, aberrations are usual. The present invention also offers a solution to this problem. As the nematodes described in this invention express marker genes at a certain growth stage, the nematodes in a culture at the same growth stage can easily be detected and isolated prior to the HTS assay. Moreover several machines are presently available that allow to select automatically nematodes which have common features (such as expressing a green fluorescent proteins). An example of such machine, generally designated as a worm dispensers or FANS (Fluorescence Activated Nematode Sorter), is provided by UBI (Union, Biometrica, USA). The methods allows the inventors to select nematodes which are in a specific growth stage, such growth stage may for example be, eggs, L1, L2, L3,L4, Adult or dauer growth stage.

In another aspect, the invention relates to the use of a (sample of at least one) nematode worm, which nematode worm contains a marker gene operably linked to a promoter, which promoter is capable of driving the expression of the marker gene in the nematode worms such that the marker gene is not expressed in at least a first development stage of the nematodes, but is expressed in at least a second development stage of the nematodes (different from the first life stage), in a method or assay for determining the influence of at least one exogenous factor on the development and/or growth on a nematode worm.

In a particular aspect, the invention relates to the use of a (sample of at least one) nematode worm in an FPTP assay as described above.

The invention will now be further illustrated by means of the following non-limiting Figures and Examples. The Figures show:

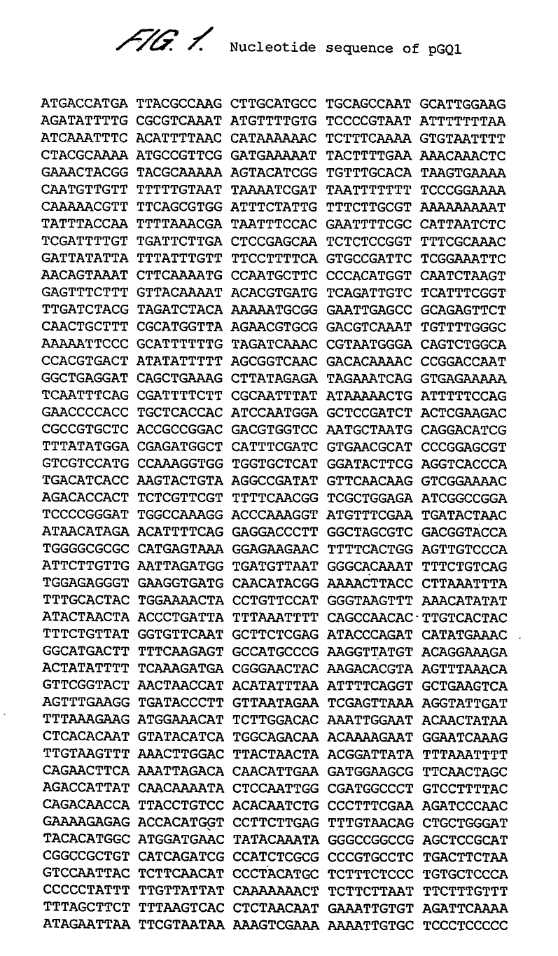

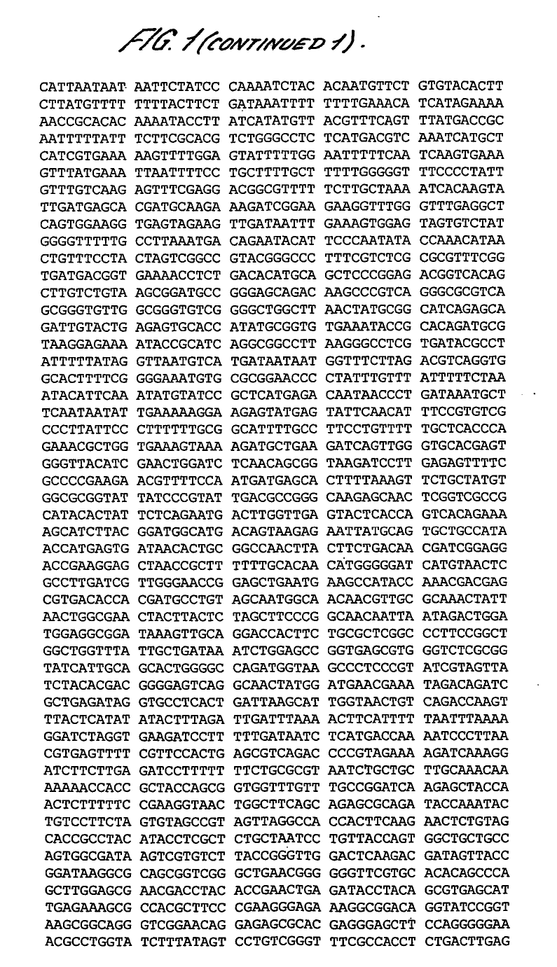

FIG. 1: Nucleotide sequence of pGQ1

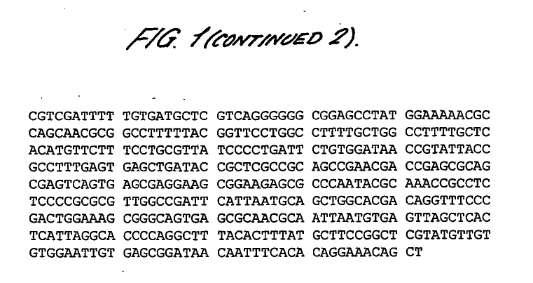

FIG. 2: Nucleotide sequence of PCLUC6

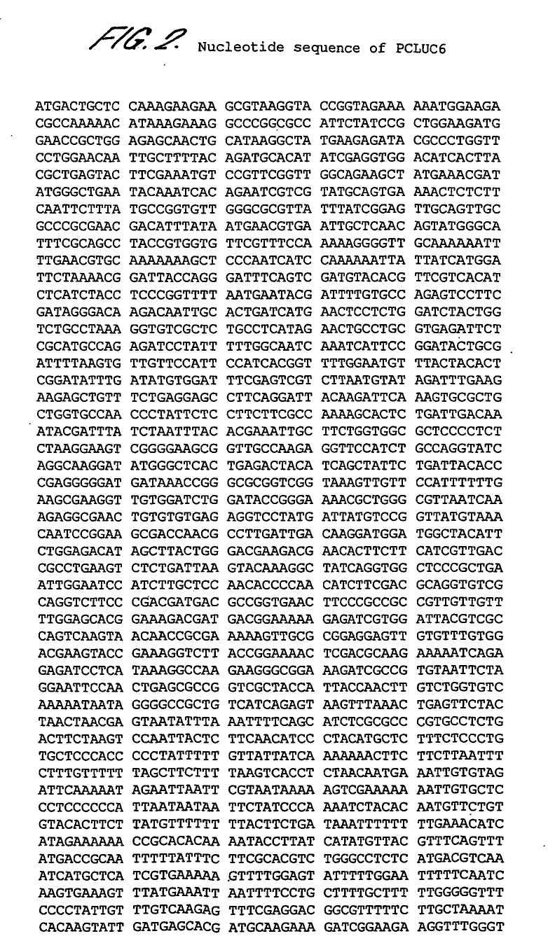

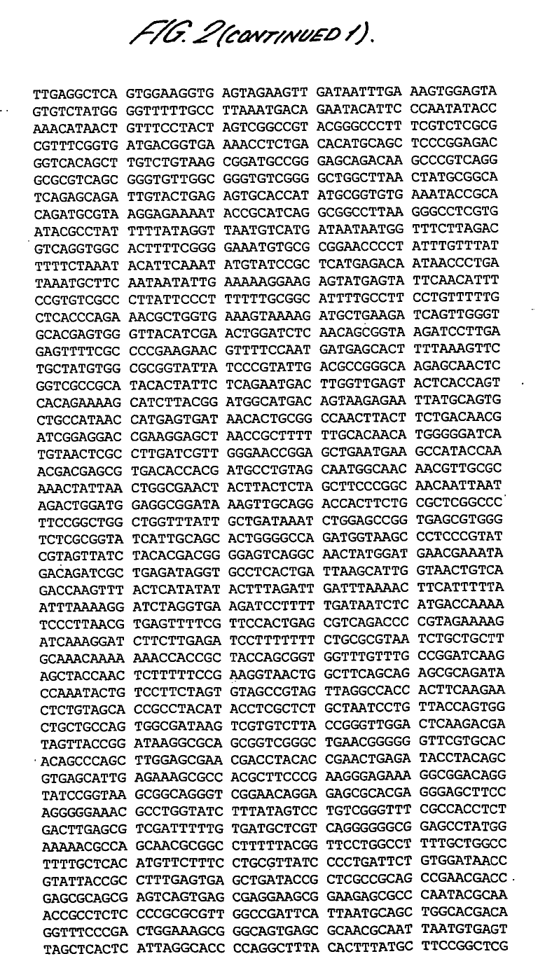

FIG. 3:Nucleotide sequence of pGQ2

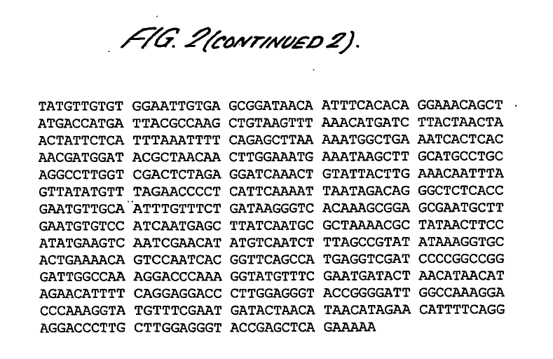

FIG. 4: Nucleotide sequence of pGN156

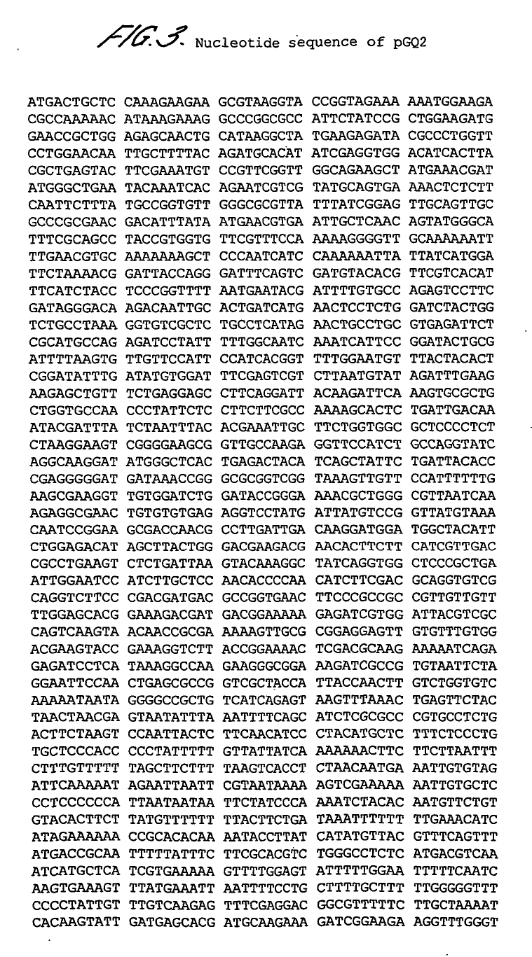

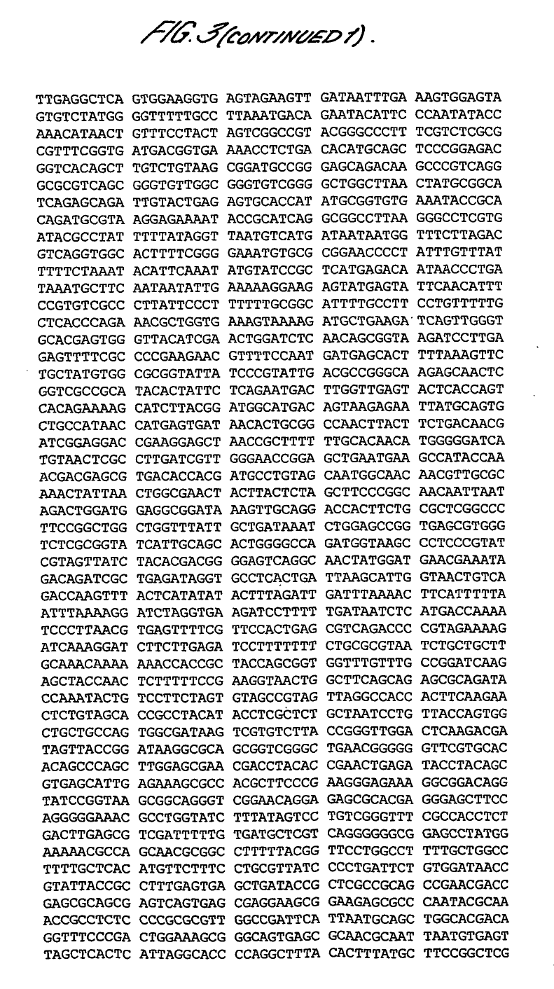

FIG. 5: Nucleotide sequence of pGQ3

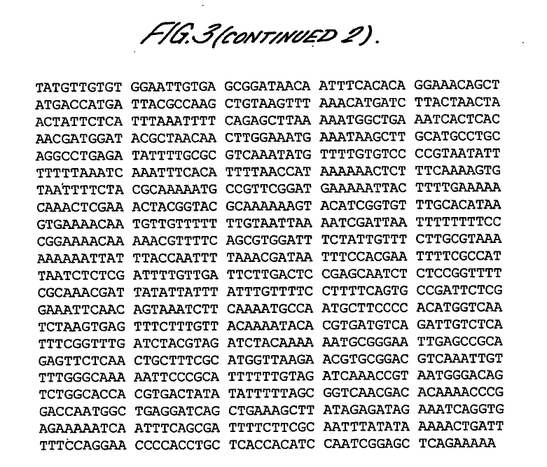

FIG. 6: Nucleotide sequence of pGQ4

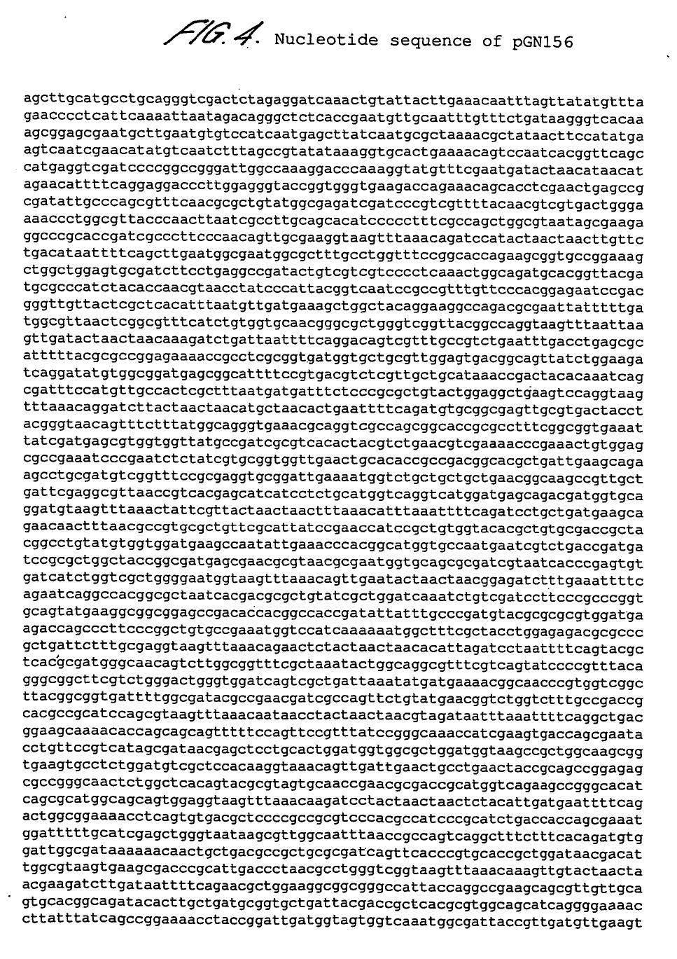

FIG. 7: Nucleotide sequence of the vit-2 promoter/NLS as present plasmid pPM143

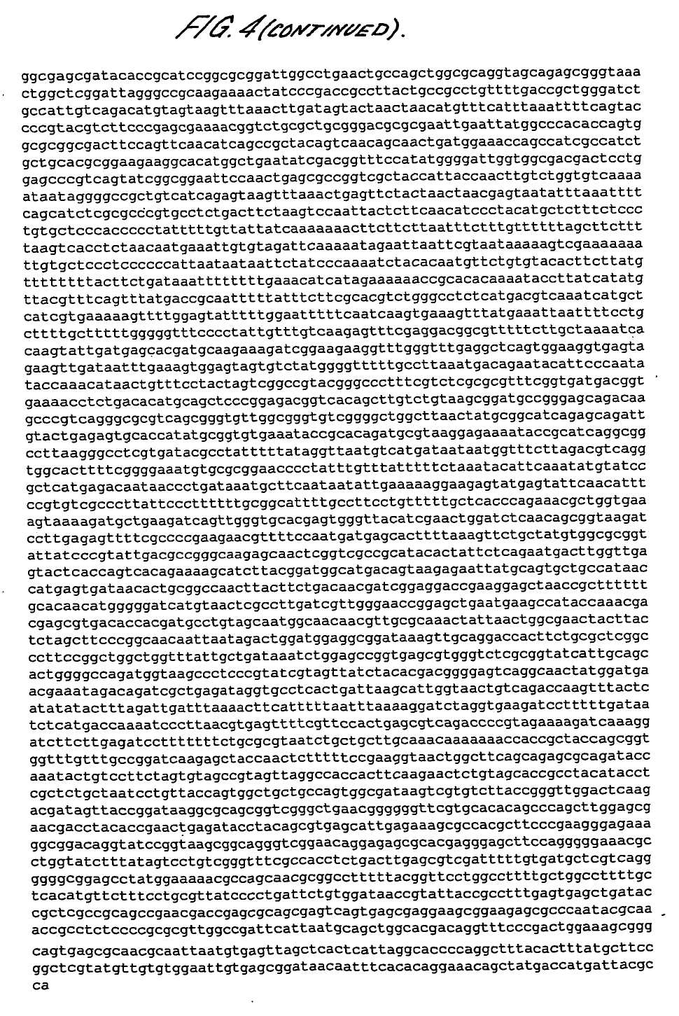

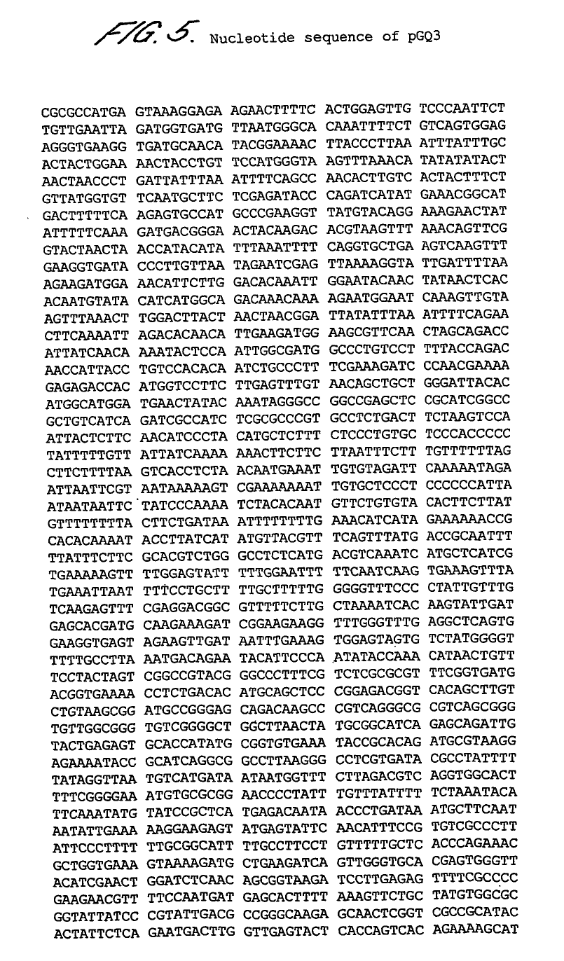

FIG. 8: Schematic drawing of pGN156

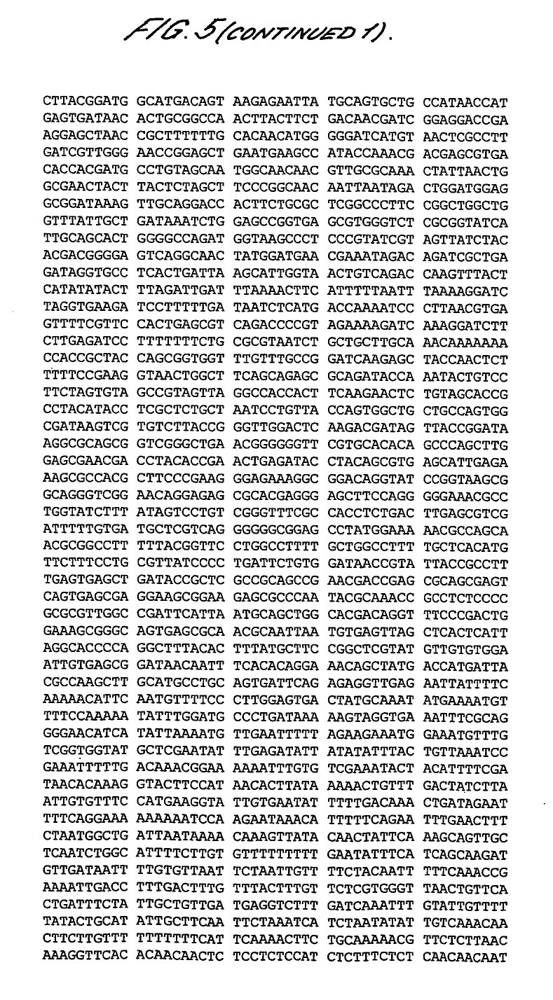

FIG. 9: Schematic drawing of pGQ1

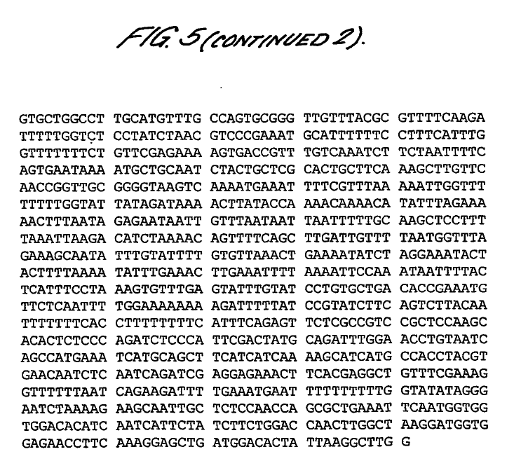

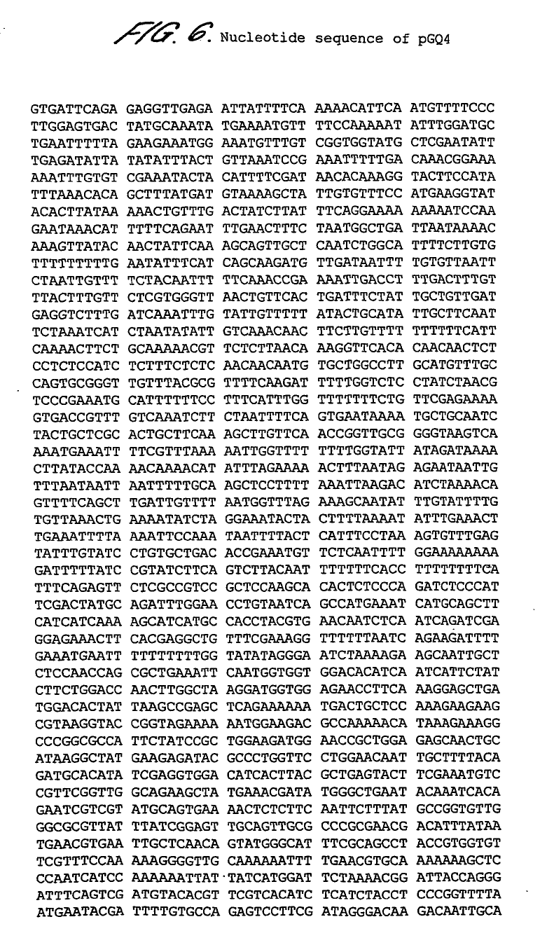

FIG. 10: Schematic drawing of pGQ2

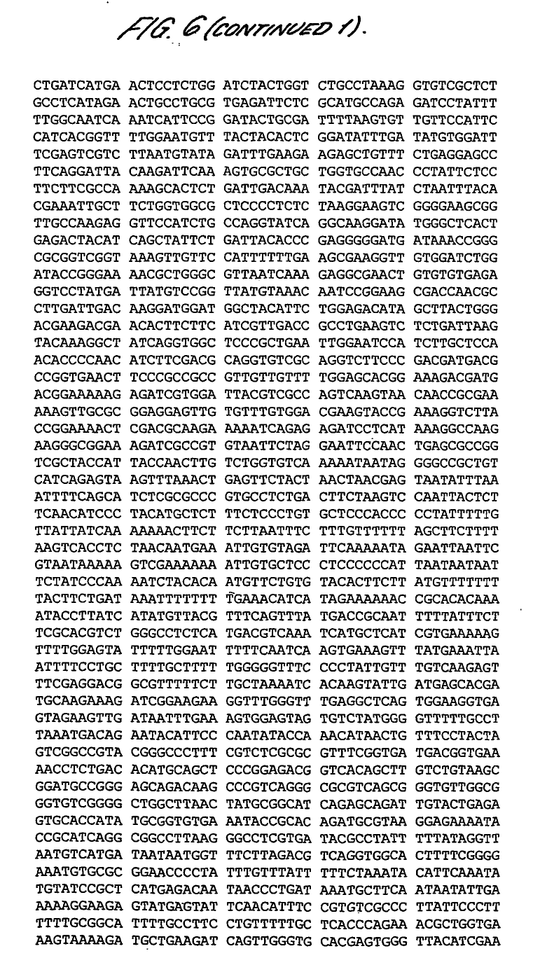

FIG. 11: Schematic drawing of pCLUC6

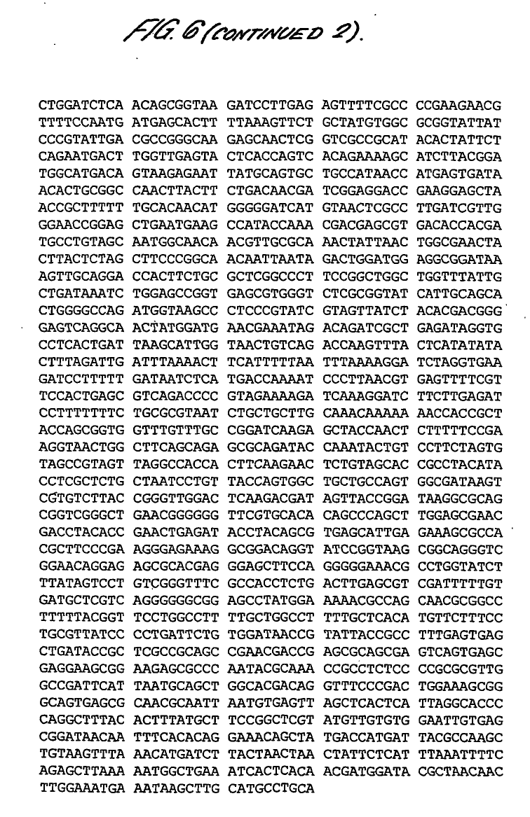

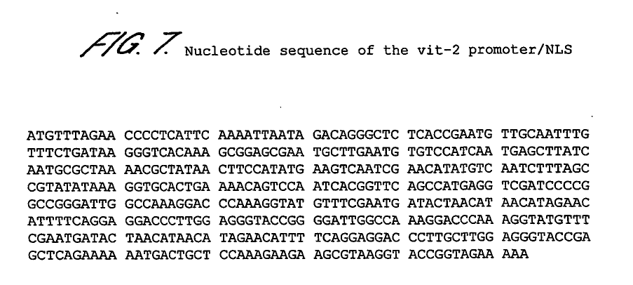

FIG. 12: Schematic drawing of pGQ3

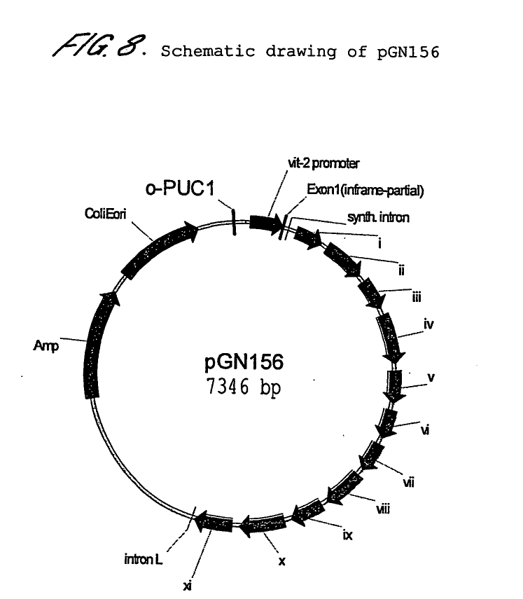

FIG. 13: Schematic drawing of pGQ4

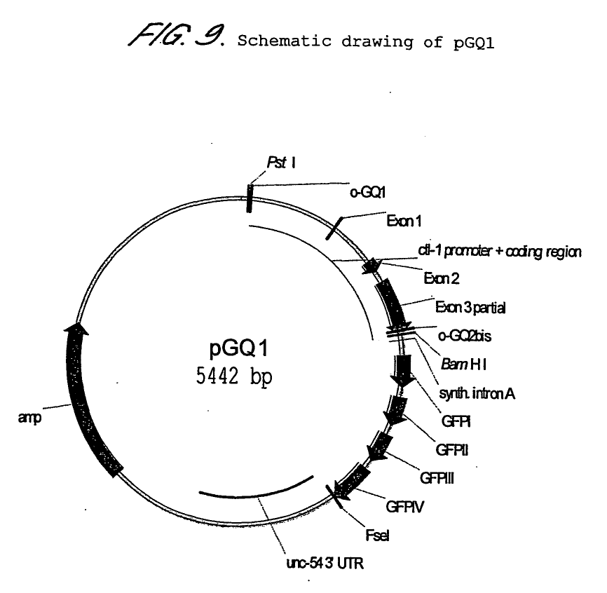

FIG. 14: Stage specific expression of LacZ (C. elegans harboring pGN156) after one hour of probe addition.

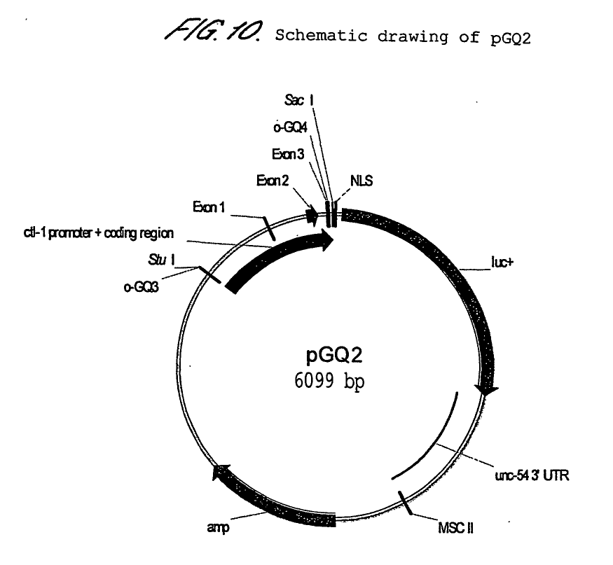

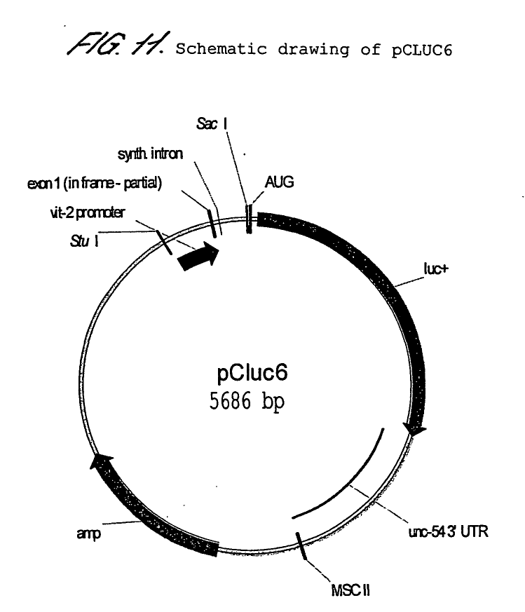

FIG. 15 : Stage specific expression of LacZ (C. elegans harboring pGN156) after two hours of probe addition.

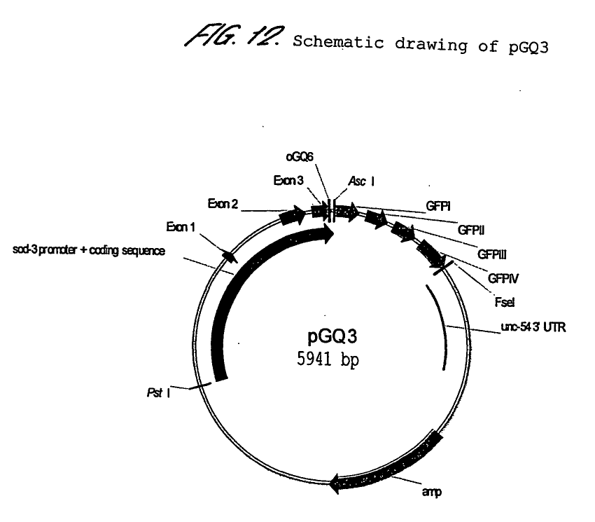

FIG. 16: Stage specific expression of LacZ (C. elegans harboring pGN156) after three hours of probe addition.

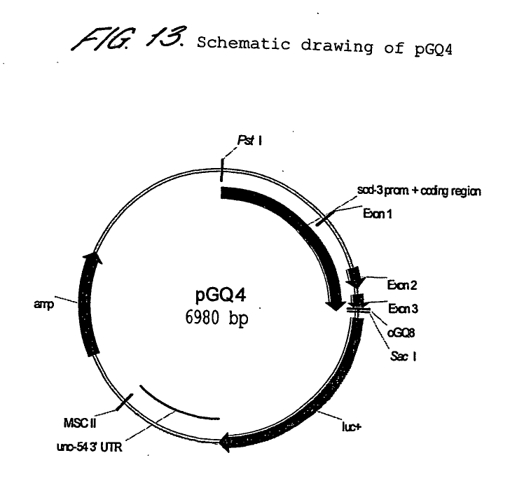

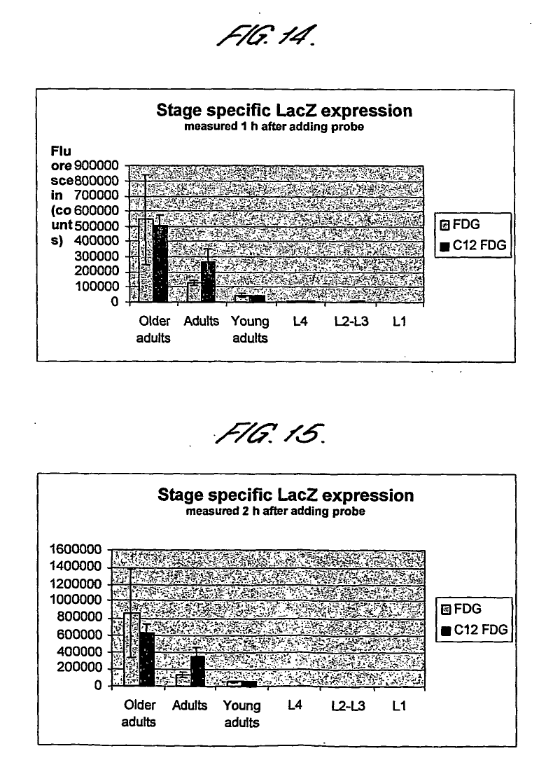

FIG. 17: Expression of Lacz in function of the number of nematodes (C. elegans harboring pGN156).

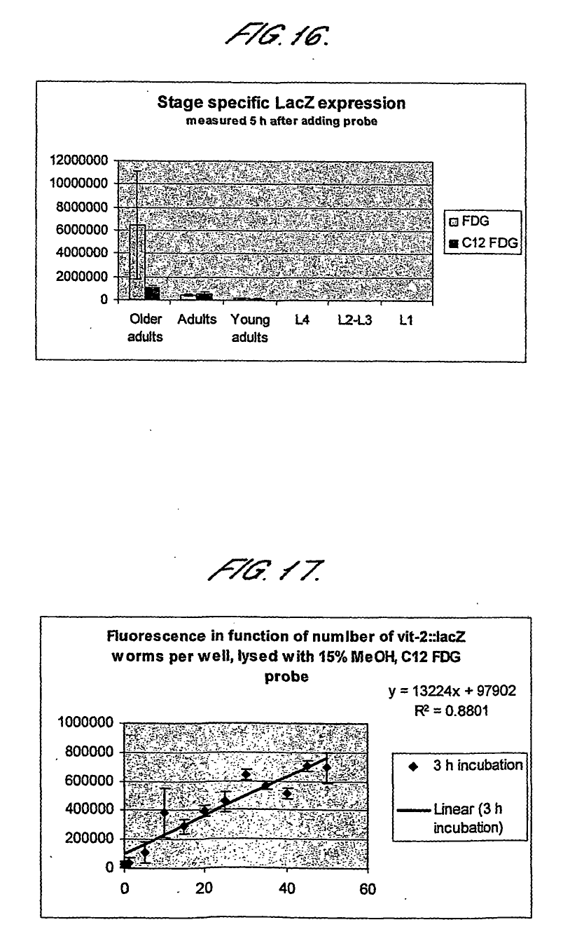

FIG. 18: Fluorescence activity of adult nematodes (C. elegans UG1513)in flat bottom wells in function of the number of wells

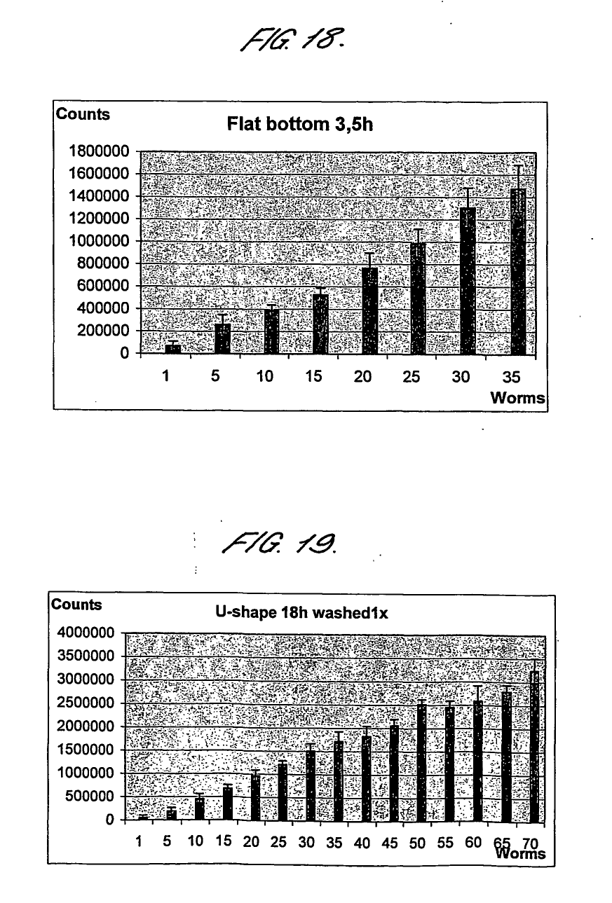

FIG. 19: Fluorescence activity of adult nematodes (C. elegans UG1513)in U-Shaped wells in function of the number of wells

Strain C. elegans UG1 353 (pGN1 56) is deposited under accession number: “LMBP 5719CB”, at the Belgian Coordinated Collection of Microorganisms (BCCM), Laboratorium voor molecular Biology-plasmidencollectie (LMBP) University of Ghent, K. L. Ledeganckstaat 35, 9000 Ghent; Belgium, according to the Budapest treaty of Apr. 28, 1977 on the international recognition of the deposit of microorganisms for the purpose of patent procedures.

### EXAMPLES

### Example 1

### Construction of Plasmids Which Allow for the Expression of Markers in a Specific Growth Stage

1) Construction of pGQ1 (ctl-1::GFP Vector) (FIGS. 1, 9)

PCR was performed on genomic DNA isolated from C. elegans wild-type strain N2 under standard conditions with following primers:

oGQ1:
5′AAAACTGCAGCCAATGCATTGGAAGAGATATTTTGCGCGTCAAATATG
TTTTGTGTCC3′
oGQ2:
5′CGCGGATCCGGCCGATTCTCCAGCGACCG3′

The PCR fragment was isolated and cloned as a PstI/BamHI fragment in pDW2020, resulting in pGQ1.

2) Construction of pGQ2 (ctl-2::Luciferase Vector) (FIGS. 3, 10)

PCR was performed on genomic DNA isolated from C. elegans wild-type strain N2 under standard conditions with following primers:

oGQ3:
5′CCAGGCCTGAGATATTTTGCGCGTCAAATATGTTTTGTGTCC3′
oGQ4:
5′CGGAGCTCCGATTGGATGTGGTGAGCAGG3′

The PCR fragment was isolated and cloned as a StuI/SacI fragment in pCIuc6, resulting in pGQ2.

3) Construction of pGQ3 (sod-3::GFP Vector) (FIG. 5, 12)

PCR was performed-on genomic DNA isolated from C. elegans wild-type strain N2 under standard conditions with following primers:

| | |
|---|---|
| oGQ7: | 5′GCAGAATTTGCAAAACGAGCAGGAAAGTC3′ |
| oGQ6: | 5′TTGGCGCGCCAAGCCTTAATAGTGTCCATCAGC3′ |

The PCR fragment was isolated and cloned as a PstI/AscI fragment in pDW2020, resulting in pGQ3.

4) Construction of pGQ4 (sod-3::Luciferase Vector) (FIGS. 6, 13)

PCR was performed on genomic DNA isolated from C. elegans wild-type strain N2 under standard conditions with following primers:

| | |
|---|---|
| oGQ7: | 5′GCAGAATTTGCAAAACGAGCAGGAAAGTC3′ |
| oGQ8: | 5′CTGAGCTCGGCTTAATAGTGTCCATCAGC3′ |

The PCR fragment was isolated and cloned as a PstI/CacIII fragment in pCIuc6, resulting in pGQ4.

5) Construction of pCIuc6 (vit-2::Luciferase Vector) (FIGS. 2, 11)

PCR was performed on genomic DNA isolated from C. elegans wild-type strain N2 under standard conditions with following primers:

vit-2F:
5′CCCCCAAGCTTCCATGTGCTAGCTGAGTTTCATCATGTCC3′
vit-2R:
5′CCCCCCAAGCTTGGCTGAACCGTGATTGG3′

The PCR fragment was isolated and cloned as a HindIII fragment in pCIuc2, resulting in pCIuc6.

6) Construction pGN156 (vit-2::lacZ Vector) (FIGS. 4, 8)

The LacZ fragment of pPD95.4 (Fire et al, Gene Gene. Sep. 14, 1990;93(2):189-98) was isolated as a SfuI/SpeI fragment and cloned in pPM143 (MacMorris et al., Gene expression vol. 3 no. p27, 1993) digested with the same enzymes, resulting in vector pGN156.

### Example 2

### Construction of C. elegans Nematodes Harboring the Plasmids Described Above, and Construction of Stable Integrated Lines

Each of the vectors was injected into C. elegans nematode worms using standard techniques as described in one of the references above. All the constructed transgenic strains showed the desired marker gene expression pattern, in a heritable way. Stable integrated line were constructed, an example is given for the integration of pGN156 (vit-2::lacZ):

1) C. elegans wild-type N2 nematodes have been injected with various concentrations of pGN156, reference and selection plasmid pGR6 (myo2::GFP), and carrier DNA (pUC18)

2) A good heritable strain was selected from the injection with 25 ng pGN156, 5 ng pGR6, 80 ng pUC18. Approximately 60 animals were gamm-irradiated (3000 rad;. 16×16 cm², 50 cm, 82.2 min) after which each worm was placed on a single plate and allowed to growth for offspring growth. Approximately 560 F1 offspring worms expressing GFP were placed each on a single plate, and allowed to grow. From The F2 generation, worms were again placed on single plates, and finally the F3 generation was checked for its GFP expression. The strains were then out-crossed with wild-type strain N2 to eliminate undesirable mutations, and checked for LacZ expression.

3) 6 selected nematodes, wherein pGN156 is integrated, were grown. From each culture, 10 nematodes were placed in the well of a 96 well plate, 25 μl M9 buffer (see above), 25 μl 60% ice cold Methanol, and 50 μl 20 mM C12FDG probe(molecular probes) was added, the wells were further incubated for 2 h at 37° C. and fluorescence was measured in a plate reader with following settings: ex/em: 485 nm/535 nm

4) One of the six strains showed high viability, strong GFP expression and relatively high LacZ expression and was selected for further analysis

This strain, designated C. elegans UG1353 (pGN156) is deposited under accession number: “LMBP 5719CB”, at the Belgian Coordinated Collection of Microorganisms (BCCM), Laboratorium voor molecular Biology-plasmidencollectie (LMBP) University of Ghent, K. L. Ledeganckstaat 35, 9000 Ghent, Belgium, according to the Budapest treaty of Apr. 28, 1977 on the international recognition of the deposit of microorganisms for the purpose of patent procedures.

### Example 3

### LacZ-Staining of an Increasing Number of C. elegans UG1353 (pGN156)

Transgenic nematodes, in various quantities per well, were dispensed using a worm dispenser: Copas 250NF (UBI), and the volume was added up to 35 μL with M9 buffer. 35 μL C12FDG (molecular probes) and 35 μL 45% methanol was added. The wells were further incubated for at least 1 h at 37° C. Fluorescence was measured with a Wallac Victor2 plate reader at ex/em: 485 nm/535 nm.

As shown in FIG. 18 and FIG. 19, the expression pattern of the transgenic nematodes is stable, which is clear from the linear increase of fluorescence versus a linear increase of nematodes in the wells.

### Example 4

### LacZ Staining of C. elegans Harboring pGN156 at Various Growth Stages

The expression pattern in function of the growth stage was measured. C. elegans harboring pGN156 was grown at various growth stages. Approximately 35 nematodes at various growth stages were placed in the wells of a microtiter plate. Each well contains only nematodes at a defined growth stage, being L1, L2-L3, L4, young adults, adults and older adults. M9 medium is added to a final volume of 35 μL.

35 μL 45% methanol and 35 μL 60 μM probe is added. Two probes have been tested:

1) Fluorescein di-beta-D-galactopyranoside (FDG) (Molecular Probes)

2) ImaGene green TM C12FDG (FDG) (Molecular Probes)

The probe was incubated for different time intervals (1 to 5 hours) at 37° C., after which the plates are cooled down to 30° C. prior to measurement.

Measurement of fluorescence was performed described above. The results are shown in FIGS. 14, 15, and 16, and clearly show that the marker gene under the control of the vit-2 promoter is only expressed at the adult growth stage.

Further more linear relationship has also been tested between the number of worms added to the well and the fluorescence measured. Essential this has been performed in the same way as described above. FIG. 17 shows the results, and the clear linearity-between the number of nematodes and the fluorescence.

### Example 5

### Constructing Mutant Strains Harboring the Integrates pGN156

The integrated pGN156 in C. elegans UG1353 can be crossed in any desired mutant available (as provided by the references above, or by the CGC, university of Minnesota, St. -Paul), or in any mutant newly created. As an example the integrated line has been crossed in a Daf-2 mutant line.

Strain UG1353 was crosses with a Wild-type male (N2) resulting in heterozygote males and hermaphrodites. A daf-2 (m41) strain was crossed with the herterozygote strain isolated above. From the offspring, the GFP expressing nematodes were isolated, and allowed self-fertilization, once again, L4 stage nematodes were isolated which express GFP, and the nematodes were placed at 25° C. to allow o form dauers. Dauers were isolated and further incubated at 15° C. The offspring was analysed and nematodes which have a 100% GFP expressing offspring are isolated for further analysis. These analysed nematodes are homozygote for both the integration of PGN156 and for daf-2 (mp41)

### Example 6

### Screening for Compounds that Affect Dauer Formation Using the daf-2 (PNZ1 56) Nematodes of the Example Above

The C. elegans daf-2 (pNZ156) nematodes were synchronized, and approximately 50 nematodes at the L1 stage were placed in each well of a 96 well plat . S medium was added as well as E. coli as described above to a final volume of 50 μL. Compound was added at a final concentration of 30 μL and th enematocdes were allowed to grow between 22° C. and 25° C. for approximately 4 days, dependent on the temperature chosen.

Methanol and probe was than added as described in the examples above to allow the detection of the expression of the LacZ marker, and the wells were further incubated for 1 hour to overnight as described above, after which the fluorescence was measured, as described above.

At a temperature higher than 22° C. this strains enters the dauer stage, at which stage no vit-2 expression, and hence no LacZ expression can be observed. Compounds which allow the nematode to bypass the dauer stage, and hence allows the nematodes to growth till the adult stage, will result in the expression of lacZ. Hence, fluorescence in detected in the wells where nematodes have been grown till at least the adult stage, hereby selecting a compound that affect dauer formation.

### Example 7

### Selection of Synchronized Worms

### Example 8

### Selection of Mutants

Chemical mutagenesis has been described extensively in C. elegans, Modem biological analysis of an organism, Methods in Cell Biology, Vol 48. Transposon mutagenesis has been described in WO 00/73510 (PCT/US00/40091). In general, the desired mutated nematodes are selected which have a desired phenotype by microscopy. When these mutagenesis techniques are performed with transgenic strains harboring a marker gene such as GFP under the control of a growth stage specific promoter, this allows for a faster and automated selection.

In Short:

Approximately 1000000 eggs of a strain harboring a marker gene under the regulation of a growth stage dependent promoter (such as vit-2::GFP) are grown till L4-young adult stage after which they are treated with the mutagen. They are allowed to growth further on plates (approximately 25.000 worms per plate). The nematodes are washed off the plates with M9 buffer, while the eggs (harboring the mutants) are allowed to grow further. The L1 offspring is then washed off and filtered using a 20 μM nylon membrane (millipore).

The L1nematodes (F1) contain the desired dominant mutants. Depending on the desired phenotype, between 2 and 50 worms are then placed in the wells of a 96 well plate, and allowed to grow further. The plates are place into a plate reader at various time intervals (approximately every 12 hours) to check the growth speed. As mutants are known to have a slower growth speed, selection can be made automatically the mutants that grow slower or selected for further analysis.

To select for recessive mutants, the L1 nematodes (F1) are allowed to grow further, and the resulting young adults are placed (approximately 500 per plate) on plates.

The eggs are isolated as above and allow to grow further till L1 stage (F2) prior to the dispensing of the nematodes into the wells, as described above. The selection occurs as described for the F1 generation.

### Example 9

### Selection in Resistance Genetics

A particular kind of mutants to be selected, are those mutants who show resistance to a compound. The addition of an active compound to a nematode result mainly in growth delay, growth arrest, lethality, and/or paralysis. Analogous as in the assay described above, mutant nematodes can be isolated that are resistant to the compound. Such mutant can be selected as the mutants will overcome the induction of the phenotype induced by the compound, and hence growth faster that the none mutated nematodes. The mutagenesis is performed as described above, while the assay and the outcome is different. In the well plates, were the L1 nematodes are allowed to grow, the compound is added. The concentration is dependent on the compound and may be between 10 μM and 350 μM, preferably 100 μM. As such compound resistance mutants will grow faster than the non-mutated nematodes, selection of the desired mutants occurs by selecting the nematodes that show firstly expression, which also has been done automatically.

### Example 10

### Growth Monitoring in RNAi Screens

Analogous to the mutatagenesis methods above, dsRNA inhibition can be performed. The principle of HTS RNAi has bee described in WO 00/01846, Nematodes can be feed by bacteria that express high amounts of dsRNA. Such RNA crossed the gut barrier, and enters the cells of C. elegans performing is RNA inhibitory action.

In Short:

Approximately 3 to 5 L1 synchronized nematodes (harboring a marker gene under the regulation of stage specific promoter) are placed in the wells of a microtiter plate, in which also E. coli bacteria are present that express high levels of dsRNA. The nematodes are allowed to grow, and those are selected that show lethality, growth delay, growth arrest, etc, which can automatically be measured as these nematodes will not enter the growth stage that allows the expression of the marker gene. The assay to select for the desired E. coli (harboring dsRNA expression of the gene of interest) is essential the same as the assu described above for mutagenesis.

In addition, a compound that induced growth delay, growth arrest, paralysis, or lethality can be added to the wells, at appropriate concentrations as described above. RNAi action on the nematode can induce resistance to such compound, analogous as has been described above for compound resistance selection. Also in this case, the nematodes are selected that overcome the phenotype induced by the compound, as they will grow faster than the nematodes that have not acquired resistance by the RNAi. As the nematodes harbor a functional promoter marker fusion, such as vit-2::GFP, only the nematodes that grow (fast), will express the marker, and hence can be selected.

1. Method for determining the influence of at least one exogenous factor on the development and/or growth of a sample of nematode worms, said method comprising:
a) providing a sample of nematode worms,
in which said nematode worms contain a marker gene operably linked to a promoter,
which promoter is capable of driving the expression of the marker gene in the nematode worms such that the marker gene is not expressed in at least a first development stage of the nematodes, but is expressed in at least a second development stage of the nematodes (different from the first life stage);
b) exposing said sample of nematode worms to at least one exogenous factor;
c) maintaining/cultivating said sample of nematode worms in a suitable medium, optionally over one or more life stages and/or generations;
d) subjecting the sample of nematode worms to at least one detection technique that is capable of detecting the signal generated by the marker gene (if expressed).

2. Method for determining the influence of at least a first exogenous factor on the development and/or growth of a sample of nematode worms, said method comprising:
a) providing at least a first and a second sample of nematode worms,
in which the nematode worms in each sample contain a marker gene operably linked to a promoter,
which promoter is capable of driving the expression of the marker gene in the nematode worms such that the marker gene is not expressed in at least a first development stage of the nematodes, but is expressed in at least a second development stage of the nematodes (different from the first life stage);
b) exposing at least said first sample of nematode worms to said first one exogenous factor;
c) maintaining/cultivating said samples of nematode worms in a suitable medium, optionally over one or more life stages and/or generations;
d) subjecting the samples of nematode worms to at least one detection t chnique that is capable of detecting the signal generated by the marker gene (if expressed);
e) determining the time required for the first sample of nematode worms to show expression of the marker gene (as determined by the signal detected for the first sample in step b)), and preferably also determining the time required for the second sample of nematode worms to show expression of the marker gene (as determined by the signal detected for the second sample in step b)); and/or comparing the time required for the first sample of nematode worms to show expression of the marker gene with the time required for the second sample of nematode worms to show expression of the marker gene.

3. Method according to claim 2, in which the second sample of nematode worms is not exposed to any exogenous factor.

4. Method according to claim 2, in which the second sample of nematode worms is exposed to a second exogenous factor.

5. Method according to any of the preceding claims, in which nematodes used are preferably from the genus Caenorhabditis, such as from Caenorhabditis briggsae or Caenorhabditis elegans.

6. Method according to claim any of the preceding claims, in which the first development stage is chosen from eggs, an embryonal stage, L1, L2 and dauer.

7. Method according to claim any of the preceding claims, in which the first development stage is L1.

8. Method according to claim any of the preceding claims, in which the first development stage is chosen from L4, adult or dauer.

9. Method according to any of the preceding claims, in which the promoter chosen from any one of the following promoters: gpl-1, unc-54, myo-2, lin-28, lin-4, lin-14, col-7, col-19, col-17, ctl-1, sod-3, vit-2.

10. Method according to any of the preceding claims, in which the promoter is the vit-2 promoter.

11. Method according to any of the preceding claims, in which marker gene is chosen from green fluorescent protein, beta-galactosidase, beta-lactamase, luciferase, acetohydroxyacid synthase, alkaline phosphatase, beta-glucuronidse, chloramphenicol acetyltransferase, horseradish peroxidase, nopaline synthase and/or octapine synthase.

12. Method according to any of the preceding claims, in which marker gene encodes a gene product that is toxic (e.g. lethal) to the nematode.

13. Method according to any of the preceding claims, in which step d) is carried out using a non-visual detection technique.

14. Method according to any of the preceding claims, which is carried out in multi-well plate format.

15. Method according to any of the preceding claims, which is carried out in an automated fashion.

16. Method according to any of the preceding claims, in which the at least one exogenous factor is at least one small compound or at least one small peptide.

17. Method according to any of the preceding claims, in which the at least one exogenous factor is a double stranded RNA sequence, suitable or intended for suppression the expression of at least one nucleotide sequence in the nematode worm by means of RNA interference.

18. Method according to any of the preceding claims, in which the nematode worms have been subjected to mutagenesis prior to use in step a).

19. Use of a (sample of at least one) nematode worm, which nematode worm contains a marker gene operably linked to a promoter, which promoter is capable of driving the expression of the marker gene in the nematode worms such that the marker gene is not expressed in at least a first development stage of the nematodes, but is expressed in at least a second development stage of the nematodes (different from the first life stage), in a method or assay for determining the influence of at least one exogenous factor on the development and/or growth on a nematode worm.

20. Use according to claim 19, in which nematodes used are preferably from the genus Caenorhabditis, such as from Caenorhabditis briggsae or Caenorhabditis elegans.

21. Use according to claim 19 or 20, in which the promoter chosen from any one of the following promoters: gpl-1, unc-54, myo-2, lin-28, lin-4, lin-14, col-7, col-19, col-17, ctl-1, sod-3, vit-2.

22. Use according to any of claims 19-21, in which the promoter is the vit-2 promoter.

23. Use according to any of claims 19-22, in which the marker gene is chosen from green fluorescent protein, beta-galactosidase, beta-lactamase, luciferase, acetohydroxyacid synthase, alkaline phosphatase, beta-glucuronidse, chloramphenicol acetyltransferase, horseradish peroxidase, nopaline synthase and/or octapine synthase.

24. Use according to any of claims 19-22, in which the marker gene encodes a gene product that is toxic (e.g. lethal) to the nematode.


SEQUENCE LISTING (7 sequences)

SEQ ID NO: 1 - DNA, 5442 bp, Artificial Sequence
  Description of Artificial Sequence Plasmid pGQ1
atgaccatgattacgccaagcttgcatgcctgcagccaatgcattggaagagatattttgcgcgtcaaatatgttttgtgtccccgtaatatttttttaaatcaaatttcacattttaaccataaaaaactctttcaaaagtgtaattttctacgcaaaaatgccgttcggatgaaaaattacttttgaaaaacaaactcgaaactacggtacgcaaaaaagtacatcggtgtttgcacataagtgaaaacaatgttgtttttttgtaattaaaatcgattaattttttttcccggaaaacaaaaacgttttcagcgtggatttctattgtttcttgcgtaaaaaaaaattatttaccaattttaaacgataatttccacgaattttcgccattaatctctcgattttgttgattcttgactccgagcaatctctccggttttcgcaaacgattatattatttatttgttttccttttcagtgccgattctcggaaattcaacagtaaatcttcaaaatgccaatgcttccccacatggtcaatctaagtgagtttctttgttacaaaatacacgtgatgtcagattgtctcatttcggtttgatctacgtagatctacaaaaaatgcgggaattgagccgcagagttctcaactgctttcgcatggttaagaacgtgcggacgtcaaattgttttgggcaaaaattcccgcattttttgtagatcaaaccgtaatgggacagtctggcaccacgtgactatatatttttagcggtcaacgacacaaaacccggaccaatggctgaggatcagctgaaagcttatagagatagaaatcaggtgagaaaaatcaatttcagcgattttcttcgcaatttatataaaaactgatttttccaggaaccccacctgctcaccacatccaatggagctccgatctactcgaagaccgccgtgctcaccgccggacgacgtggtccaatgctaatgcaggacatcgtttatatggacgagatggctcatttcgatcgtgaacgcatcccggagcgtgtcgtccatgccaaaggtggtggtgctcatggatacttcgaggtcacccatgacatcaccaagtactgtaaggccgatatgttcaacaaggtcggaaaacagacaccacttctcgttcgtttttcaacggtcgctggagaatcggccggatccccgggattggccaaaggacccaaaggtatgtttcgaatgatactaacataacatagaacattttcaggaggacccttggctagcgtcgacggtaccatggggcgcgccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatgggtaagtttaaacatatatatactaactaaccctgattatttaaattttcagccaacacttgtcactactttctgttatggtgttcaatgcttctcgagatacccagatcatatgaaacggcatgactttttcaagagtgccatgcccgaaggttatgtacaggaaagaactatatttttcaaagatgacgggaactacaagacacgtaagtttaaacagttcggtactaactaaccatacatatttaaattttcaggtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttgtaagtttaaacttggacttactaactaacggattatatttaaattttcagaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagggccggccgagctccgcatcggccgctgtcatcagatcgccatctcgcgcccgtgcctctgacttctaagtccaattactcttcaacatccctacatgctctttctccctgtgctcccaccccctatttttgttattatcaaaaaaacttcttcttaatttctttgttttttagcttcttttaagtcacctctaacaatgaaattgtgtagattcaaaaatagaattaattcgtaataaaaagtcgaaaaaaattgtgctccctccccccattaataataattctatcccaaaatctacacaatgttctgtgtacacttcttatgttttttttacttctgataaattttttttgaaacatcatagaaaaaaccgcacacaaaataccttatcatatgttacgtttcagtttatgaccgcaatttttatttcttcgcacgtctgggcctctcatgacgtcaaatcatgctcatcgtgaaaaagttttggagtatttttggaatttttcaatcaagtgaaagtttatgaaattaattttcctgcttttgctttttgggggtttcccctattgtttgtcaagagtttcgaggacggcgtttttcttgctaaaatcacaagtattgatgagcacgatgcaagaaagatcggaagaaggtttgggtttgaggctcagtggaaggtgagtagaagttgataatttgaaagtggagtagtgtctatggggtttttgccttaaatgacagaatacattcccaatataccaaacataactgtttcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagct

SEQ ID NO: 2 - DNA, 5686 bp, Artificial Sequence
  Description of Artificial Sequence Plasmid pcLUC6
atgactgctccaaagaagaagcgtaaggtaccggtagaaaaaatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctaggaattccaactgagcgccggtcgctaccattaccaacttgtctggtgtcaaaaataataggggccgctgtcatcagagtaagtttaaactgagttctactaactaacgagtaatatttaaattttcagcatctcgcgcccgtgcctctgacttctaagtccaattactcttcaacatccctacatgctctttctccctgtgctcccaccccctatttttgttattatcaaaaaaacttcttcttaatttctttgttttttagcttcttttaagtcacctctaacaatgaaattgtgtagattcaaaaatagaattaattcgtaataaaaagtcgaaaaaaattgtgctccctccccccattaataataattctatcccaaaatctacacaatgttctgtgtacacttcttatgttttttttacttctgataaattttttttgaaacatcatagaaaaaaccgcacacaaaataccttatcatatgttacgtttcagtttatgaccgcaatttttatttcttcgcacgtctgggcctctcatgacgtcaaatcatgctcatcgtgaaaaagttttggagtatttttggaatttttcaatcaagtgaaagtttatgaaattaattttcctgcttttgctttttgggggtttcccctattgtttgtcaagagtttcgaggacggcgtttttcttgctaaaatcacaagtattgatgagcacgatgcaagaaagatcggaagaaggtttgggtttgaggctcagtggaaggtgagtagaagttgataatttgaaagtggagtagtgtctatggggtttttgccttaaatgacagaatacattcccaatataccaaacataactgtttcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgtaagtttaaacatgatcttactaactaactattctcatttaaattttcagagcttaaaaatggctgaaatcactcacaacgatggatacgctaacaacttggaaatgaaataagcttgcatgcctgcaggccttggtcgactctagaggatcaaactgtattacttgaaacaatttagttatatgtttagaacccctcattcaaaattaatagacagggctctcaccgaatgttgcaatttgtttctgataagggtcacaaagcggagcgaatgcttgaatgtgtccatcaatgagcttatcaatgcgctaaaacgctataacttccatatgaagtcaatcgaacatatgtcaatctttagccgtatataaaggtgcactgaaaacagtccaatcacggttcagccatgaggtcgatccccggccgggattggccaaaggacccaaaggtatgtttcgaatgatactaacataacatagaacattttcaggaggacccttggagggtaccggggattggccaaaggacccaaaggtatgtttcgaatgatactaacataacatagaacattttcaggaggacccttgcttggagggtaccgagctcagaaaaa

SEQ ID NO: 3 - DNA, 6099 bp, Artificial Sequence
  Description of Artificial Sequence Plasmid pGQ2
atgactgctccaaagaagaagcgtaaggtaccggtagaaaaaatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatttcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctaggaattccaactgagcgccggtcgctaccattaccaacttgtctggtgtcaaaaataataggggccgctgtcatcagagtaagtttaaactgagttctactaactaacgagtaatatttaaattttcagcatctcgcgcccgtgcctctgacttctaagtccaattactcttcaacatccctacatgctctttctccctgtgctcccaccccctatttttgttattatcaaaaaaacttcttcttaatttctttgttttttagcttcttttaagtcacctctaacaatgaaattgtgtagattcaaaaatagaattaattcgtaataaaaagtcgaaaaaaattgtgctccctccccccattaataataattctatcccaaaatctacacaatgttctgtgtacacttcttatgttttttttacttctgataaattttttttgaaacatcatagaaaaaaccgcacacaaaataccttatcatatgttacgtttcagtttatgaccgcaatttttatttcttcgcacgtctgggcctctcatgacgtcaaatcatgctcatcgtgaaaaagttttggagtatttttggaatttttcaatcaagtgaaagtttatgaaattaattttcctgcttttgctttttgggggtttcccctattgtttgtcaagagtttcgaggacggcgtttttcttgctaaaatcacaagtattgatgagcacgatgcaagaaagatcggaagaaggtttgggtttgaggctcagtggaaggtgagtagaagttgataatttgaaagtggagtagtgtctatggggtttttgccttaaatgacagaatacattcccaatataccaaacataactgtttcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgtaagtttaaacatgatcttactaactaactattctcatttaaattttcagagcttaaaaatggctgaaatcactcacaacgatggatacgctaacaacttggaaatgaaataagcttgcatgcctgcaggcctgagatattttgcgcgtcaaatatgttttgtgtccccgtaatatttttttaaatcaaatttcacattttaaccataaaaaactctttcaaaagtgtaattttctacgcaaaaatgccgttcggatgaaaaattacttttgaaaaacaaactcgaaactacggtacgcaaaaaagtacatcggtgtttgcacataagtgaaaacaatgttgtttttttgtaattaaaatcgattaattttttttcccggaaaacaaaaacgttttcagcgtggatttctattgtttcttgcgtaaaaaaaaattatttaccaattttaaacgataatttccacgaattttcgccattaatctctcgattttgttgattcttgactccgagcaatctctccggttttcgcaaacgattatattatttatttgttttccttttcagtgccgattctcggaaattcaacagtaaatcttcaaaatgccaatgcttccccacatggtcaatctaagtgagtttctttgttacaaaatacacgtgatgtcagattgtctcatttcggtttgatctacgtagatctacaaaaaatgcgggaattgagccgcagagttctcaactgctttcgcatggttaagaacgtgcggacgtcaaattgttttgggcaaaaattcccgcattttttgtagatcaaaccgtaatgggacagtctggcaccacgtgactatatatttttagcggtcaacgacacaaaacccggaccaatggctgaggatcagctgaaagcttatagagatagaaatcaggtgagaaaaatcaatttcagcgattttcttcgcaatttatataaaaactgatttttccaggaaccccacctgctcaccacatccaatcggagctcagaaaaa

SEQ ID NO: 4 - DNA, 7346 bp, Artificial Sequence
  Description of Artificial Sequence Plasmid pGN156
agcttgcatgcctgcagggtcgactctagaggatcaaactgtattacttgaaacaatttagttatatgtttagaacccctcattcaaaattaatagacagggctctcaccgaatgttgcaatttgtttctgataagggtcacaaagcggagcgaatgcttgaatgtgtccatcaatgagcttatcaatgcgctaaaacgctataacttccatatgaagtcaatcgaacatatgtcaatctttagccgtatataaaggtgcactgaaaacagtccaatcacggttcagccatgaggtcgatccccggccgggattggccaaaggacccaaaggtatgtttcgaatgatactaacataacatagaacattttcaggaggacccttggagggtaccggtgggtgaagaccagaaacagcacctcgaactgagccgcgatattgcccagcgtttcaacgcgctgtatggcgagatcgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgaaggtaagtttaaacagatccatactaactaacttgttctgacataattttcagcttgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggtaagtttaattaagttgatactaactaacaaagatctgattaattttcaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttctcccgcgctgtactggaggctgaagtccaggtaagtttaaacaggatcttactaactaacatgctaacactgaattttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatgtaagtttaaactattcgttactaactaactttaaacatttaaattttcagatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatggtaagtttaaacagttgaatactaactaacggagatctttgaaattttcagaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgattctttgcgaggtaagtttaaacagaactctactaactaacacattagatcctaattttcagtacgctcacgcgatgggcaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgtaagtttaaacaataacctactaactaacgtagataatttaaattttcaggctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcatggcagcagtggaggtaagtttaaacaagatcctactaactaactctacattgatgaattttcagactggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcggtaagtttaaacaaagttgtactaactaacgaagatcttgataattttcagaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtagtaagtttaaacttgatagtactaactaacatgtttcatttaaattttcagtaccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccaactgagcgccggtcgctaccattaccaacttgtctggtgtcaaaaataataggggccgctgtcatcagagtaagtttaaactgagttctactaactaacgagtaatatttaaattttcagcatctcgcgcccgtgcctctgacttctaagtccaattactcttcaacatccctacatgctctttctccctgtgctcccaccccctatttttgttattatcaaaaaaacttcttcttaatttctttgttttttagcttcttttaagtcacctctaacaatgaaattgtgtagattcaaaaatagaattaattcgtaataaaaagtcgaaaaaaattgtgctccctccccccattaataataattctatcccaaaatctacacaatgttctgtgtacacttcttatgttttttttacttctgataaattttttttgaaacatcatagaaaaaaccgcacacaaaataccttatcatatgttacgtttcagtttatgaccgcaatttttatttcttcgcacgtctgggcctctcatgacgtcaaatcatgctcatcgtgaaaaagttttggagtatttttggaatttttcaatcaagtgaaagtttatgaaattaattttcctgcttttgctttttgggggtttcccctattgtttgtcaagagtttcgaggacggcgtttttcttgctaaaatcacaagtattgatgagcacgatgcaagaaagatcggaagaaggtttgggtttgaggctcagtggaaggtgagtagaagttgataatttgaaagtggagtagtgtctatggggtttttgccttaaatgacagaatacattcccaatataccaaacataactgtttcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcca

SEQ ID NO: 5 - DNA, 5991 bp, Artificial Sequence
  Description of Artificial Sequence Plasmid pGQ3
cgcgccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatgggtaagtttaaacatatatatactaactaaccctgattatttaaattttcagccaacacttgtcactactttctgttatggtgttcaatgcttctcgagatacccagatcatatgaaacggcatgactttttcaagagtgccatgcccgaaggttatgtacaggaaagaactatatttttcaaagatgacgggaactacaagacacgtaagtttaaacagttcggtactaactaaccatacatatttaaattttcaggtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttgtaagtttaaacttggacttactaactaacggattatatttaaattttcagaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagggccggccgagctccgcatcggccgctgtcatcagatcgccatctcgcgcccgtgcctctgacttctaagtccaattactcttcaacatccctacatgctctttctccctgtgctcccaccccctatttttgttattatcaaaaaaacttcttcttaatttctttgttttttagcttcttttaagtcacctctaacaatgaaattgtgtagattcaaaaatagaattaattcgtaataaaaagtcgaaaaaaattgtgctccctccccccattaataataattctatcccaaaatctacacaatgttctgtgtacacttcttatgttttttttacttctgataaattttttttgaaacatcatagaaaaaaccgcacacaaaataccttatcatatgttacgtttcagtttatgaccgcaatttttatttcttcgcacgtctgggcctctcatgacgtcaaatcatgctcatcgtgaaaaagttttggagtatttttggaatttttcaatcaagtgaaagtttatgaaattaattttcctgcttttgctttttgggggtttcccctattgtttgtcaagagtttcgaggacggcgtttttcttgctaaaatcacaagtattgatgagcacgatgcaagaaagatcggaagaaggtttgggtttgaggctcagtggaaggtgagtagaagttgataatttgaaagtggagtagtgtctatggggtttttgccttaaatgacagaatacattcccaatataccaaacataactgtttcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagtgattcagagaggttgagaattattttcaaaaacattcaatgttttcccttggagtgactatgcaaatatgaaaatgttttccaaaaatatttggatgccctgataaaaagtaggtgaaatttcgcaggggaacatcatattaaaatgttgaatttttagaagaaatggaaatgtttgtcggtggtatgctcgaatatttgagatattatatatttactgttaaatccgaaatttttgacaaacggaaaaaatttgtgtcgaaatactacattttcgataacacaaaggtacttccataacacttataaaaactgtttgactatcttaattgtgtttccatgaaggtattgtgaatatttttgacaaactgatagaattttcaggaaaaaaaaatccaagaataaacatttttcagaatttgaactttctaatggctgattaataaaacaaagttatacaactattcaaagcagttgctcaatctggcattttcttgtgtttttttttgaatatttcatcagcaagatgttgataattttgtgttaattctaattgttttctacaatttttcaaaccgaaaattgacctttgactttgtttactttgttctcgtgggttaactgttcactgatttctattgctgttgatgaggtctttgatcaaatttgtattgtttttatactgcatattgcttcaattctaaatcatctaatatattgtcaaacaacttcttgttttttttttcattcaaaacttctgcaaaaacgttctcttaacaaaggttcacacaacaactctcctctccatctctttctctcaacaacaatgtgctggccttgcatgtttgccagtgcgggttgtttacgcgttttcaagatttttggtctcctatctaacgtcccgaaatgcattttttcctttcatttggtttttttctgttcgagaaaagtgaccgtttgtcaaatcttctaattttcagtgaataaaatgctgcaatctactgctcgcactgcttcaaagcttgttcaaccggttgcggggtaagtcaaaatgaaattttcgtttaaaaattggttttttttggtattatagataaaacttataccaaaacaaaacatatttagaaaaactttaatagagaataattgtttaataattaatttttgcaagctccttttaaattaagacatctaaaacagttttcagcttgattgttttaatggtttagaaagcaatatttgtattttgtgttaaactgaaaatatctaggaaatactacttttaaaatatttgaaacttgaaattttaaaattccaaataattttactcatttcctaaagtgtttgagtatttgtatcctgtgctgacaccgaaatgttctcaattttggaaaaaaaagatttttatccgtatcttcagtcttacaatttttttcaccttttttttcatttcagagttctcgccgtccgctccaagcacactctcccagatctcccattcgactatgcagatttggaacctgtaatcagccatgaaatcatgcagcttcatcatcaaaagcatcatgccacctacgtgaacaatctcaatcagatcgaggagaaacttcacgaggctgtttcgaaaggttttttaatcagaagattttgaaatgaattttttttttggtatatagggaatctaaaagaagcaattgctctccaaccagcgctgaaattcaatggtggtggacacatcaatcattctatcttctggaccaacttggctaaggatggtggagaaccttcaaaggagctgatggacactattaaggcttgg

SEQ ID NO: 6 - DNA, 6980 bp, Artificial Sequence
  Description of Artificial Sequence Plasmid pGQ4
gtgattcagagaggttgagaattattttcaaaaacattcaatgttttcccttggagtgactatgcaaatatgaaaatgttttccaaaaatatttggatgctgaatttttagaagaaatggaaatgtttgtcggtggtatgctcgaatatttgagatattatatatttactgttaaatccgaaatttttgacaaacggaaaaaatttgtgtcgaaatactacattttcgataacacaaaggtacttccatatttaaacacagctttatgatgtaaaagctattgtgtttccatgaaggtatacacttataaaaactgtttgactatcttatttcaggaaaaaaaaatccaagaataaacatttttcagaatttgaactttctaatggctgattaataaaacaaagttatacaactattcaaagcagttgctcaatctggcattttcttgtgtttttttttgaatatttcatcagcaagatgttgataattttgtgttaattctaattgttttctacaatttttcaaaccgaaaattgacctttgactttgtttactttgttctcgtgggttaactgttcactgatttctattgctgttgatgaggtctttgatcaaatttgtattgtttttatactgcatattgcttcaattctaaatcatctaatatattgtcaaacaacttcttgttttttttttcattcaaaacttctgcaaaaacgttctcttaacaaaggttcacacaacaactctcctctccatctctttctctcaacaacaatgtgctggccttgcatgtttgccagtgcgggttgtttacgcgttttcaagatttttggtctcctatctaacgtcccgaaatgcattttttcctttcatttggtttttttctgttcgagaaaagtgaccgtttgtcaaatcttctaattttcagtgaataaaatgctgcaatctactgctcgcactgcttcaaagcttgttcaaccggttgcggggtaagtcaaaatgaaattttcgtttaaaaattggttttttttggtattatagataaaacttataccaaaacaaaacatatttagaaaaactttaatagagaataattgtttaataattaatttttgcaagctccttttaaattaagacatctaaaacagttttcagcttgattgttttaatggtttagaaagcaatatttgtattttgtgttaaactgaaaatatctaggaaatactacttttaaaatatttgaaacttgaaattttaaaattccaaataattttactcatttcctaaagtgtttgagtatttgtatcctgtgctgacaccgaaatgttctcaattttggaaaaaaaagatttttatccgtatcttcagtcttacaatttttttcaccttttttttcatttcagagttctcgccgtccgctccaagcacactctcccagatctcccattcgactatgcagatttggaacctgtaatcagccatgaaatcatgcagcttcatcatcaaaagcatcatgccacctacgtgaacaatctcaatcagatcgaggagaaacttcacgaggctgtttcgaaaggttttttaatcagaagattttgaaatgaattttttttttggtatatagggaatctaaaagaagcaattgctctccaaccagcgctgaaattcaatggtggtggacacatcaatcattctatcttctggaccaacttggctaaggatggtggagaaccttcaaaggagctgatggacactattaagccgagctcagaaaaaatgactgctccaaagaagaagcgtaaggtaccggtagaaaaaatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctaggaattccaactgagcgccggtcgctaccattaccaacttgtctggtgtcaaaaataataggggccgctgtcatcagagtaagtttaaactgagttctactaactaacgagtaatatttaaattttcagcatctcgcgcccgtgcctctgacttctaagtccaattactcttcaacatccctacatgctctttctccctgtgctcccaccccctatttttgttattatcaaaaaaacttcttcttaatttctttgttttttagcttcttttaagtcacctctaacaatgaaattgtgtagattcaaaaatagaattaattcgtaataaaaagtcgaaaaaaattgtgctccctccccccattaataataattctatcccaaaatctacacaatgttctgtgtacacttcttatgttttttttacttctgataaattttttttgaaacatcatagaaaaaaccgcacacaaaataccttatcatatgttacgtttcagtttatgaccgcaatttttatttcttcgcacgtctgggcctctcatgacgtcaaatcatgctcatcgtgaaaaagttttggagtatttttggaatttttcaatcaagtgaaagtttatgaaattaattttcctgcttttgctttttgggggtttcccctattgtttgtcaagagtttcgaggacggcgtttttcttgctaaaatcacaagtattgatgagcacgatgcaagaaagatcggaagaaggtttgggtttgaggctcagtggaaggtgagtagaagttgataatttgaaagtggagtagtgtctatggggtttttgccttaaatgacagaatacattcccaatataccaaacataactgtttcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgtaagtttaaacatgatcttactaactaactattctcatttaaattttcagagcttaaaaatggctgaaatcactcacaacgatggatacgctaacaacttggaaatgaaataagcttgcatgcctgca

SEQ ID NO: 7 - DNA, 473 bp, Artificial Sequence
  Description of Artificial Sequence Vit-2 promoter/NLS fragment
atgtttagaacccctcattcaaaattaatagacagggctctcaccgaatgttgcaatttgtttctgataagggtcacaaagcggagcgaatgcttgaatgtgtccatcaatgagcttatcaatgcgctaaaacgctataacttccatatgaagtcaatcgaacatatgtcaatctttagccgtatataaaggtgcactgaaaacagtccaatcacggttcagccatgaggtcgatccccggccgggattggccaaaggacccaaaggtatgtttcgaatgatactaacataacatagaacattttcaggaggacccttggagggtaccggggattggccaaaggacccaaaggtatgtttcgaatgatactaacataacatagaacattttcaggaggacccttgcttggagggtaccgagctcagaaaaaatgactgctccaaagaagaagcgtaaggtaccggtagaaaaa